(19) United States
(12) Patent Application Publication
(10) Pub. No.: US 2026/0013928 A1
(43) Pub. Date: Jan. 15, 2026

(54) MULTIFUNCTIONAL LAPAROSCOPIC INSTRUMENT WITH INTEGRATED FLUID AND ENERGY DELIVERY

(71) Applicant: Sebastian A. Bernaschina-Rivera, Ponce, PR (US)

(72) Inventor: Sebastian A. Bernaschina-Rivera, Ponce, PR (US)

(21) Appl. No.: 19/263,440

(22) Filed: Jul. 8, 2025

Related U.S. Application Data
(60) Provisional application No. 63/669,410, filed on Jul. 10, 2024.

(51) Int. Cl. A61B 18/12; A61B 18/00; A61B 18/14; A61B 90/00

(52) U.S. Cl. CPC A61B 18/1206; A61B 18/1442; A61B 90/06; A61B 2018/00184; A61B 2018/00595; A61B 2018/00702; A61B 2018/00922; A61B 2018/0094; A61B 2218/002; A61B 2218/007

(57) ABSTRACT

A surgical instrument includes a handle assembly with a housing, a trigger movably coupled to the housing, a joystick movably coupled to the housing, a mode selection interface disposed on an exterior surface of the housing, and a microcontroller unit (MCU) disposed within the housing. An elongate shaft extends distally from the handle assembly. An end effector assembly is disposed at a distal end of the elongate shaft. The MCU is operably coupled to the trigger, the joystick, and the mode selection interface, and is configured to receive input signals from the trigger, the joystick, and the mode selection interface, and to control operation of the surgical instrument based on the received input signals and a selected operative mode.

### CROSS REFERENCE TO RELATED APPLICATIONS

This application claims the benefit of U.S. Provisional Patent Application No. 63/669,410, filed on Jul. 10, 2024, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all rights whatsoever.

### BACKGROUND OF THE INVENTION

### 1) Field of the Invention

The invention relates in general to the field of laparoscopic instruments, and more particularly, to laparoscopic instrument s for minimally invasive procedures, and more particularly to a multifunctional laparoscopic instrument that integrates grasping, articulation, electrosurgery, and fluid management capabilities into a single handheld device.

### 2) Description of Related Art

Currently the state of the art includes numerous Laparoscopic surgery options including Appendectomy, Cholecystectomy, Hernia repair, Bariatric surgery, Colectomy, Hepatectomy and others. The need for better and more convenient to use tools is pushing the technology and opening up new areas of practice.

Laparoscopic surgery has become increasingly prevalent in modern medical practice due to its minimally invasive nature, offering benefits such as reduced patient trauma, shorter recovery times, and improved cosmetic outcomes compared to traditional open surgery. This surgical approach involves making small incisions through which specialized instruments and a camera are inserted to perform procedures within the abdominal cavity.

Laparoscopic surgery has become a widely adopted technique due to its reduced invasiveness, shorter recovery times, and minimized scarring compared to open surgery. However, the constraints imposed by the limited number of access ports and the narrow working envelope within the abdominal cavity often necessitate multiple instrument exchanges and frequent repositioning. These factors can contribute to prolonged operative time, increased risk of tissue trauma, and reduced procedural efficiency.

Traditional laparoscopic graspers are typically limited to a singular mechanical function—namely, tissue manipulation through grasping, traction, or retraction and also may have cautery function. In procedures requiring coagulation, cutting, suction, irrigation, or sensory feedback, surgeons must employ separate instruments, thereby introducing delays and ergonomic challenges.

As laparoscopic techniques have advanced, there has been a growing demand for laparoscopic instrument s that can perform multiple functions without requiring frequent exchanges during procedures. Conventional laparoscopic tools often have limited functionality, necessitating the use of several different instruments to complete a single operation. This can lead to increased procedure duration, additional incisions, and potential complications associated with instrument exchanges.

Irrigation and suction capabilities, which are often required for maintaining a clear operative field, are usually provided by standalone instruments.

The field of laparoscopic instrumentation continues to progress, driven by the need for tools that can adapt to a wide range of surgical scenarios while maintaining simplicity of use and adherence to safety standards. Innovations in this area have the potential to further refine minimally invasive surgical techniques and expand their applicability across various medical specialties.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide multifunction laparoscopic instrumentation.

The present invention relates generally to laparoscopic instrument s used in minimally invasive procedures and, more particularly, to a multifunctional enhanced laparoscopic grasper system configured to integrate additional operative capabilities into a single grasping tool. These functionalities include irrigation, suction, electronic assisted articulation of distal ends, and electrosurgery. The device aims to provide a novel way for surgeons to conduct minimally invasive procedures, allowing for faster and safer responses during surgery.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

As surgical techniques continue to evolve, there is an ongoing effort to develop instruments that can enhance efficiency, reduce the number of required incisions, and improve overall surgical outcomes. Advancements in materials science, miniaturized electronics, and control systems offer opportunities for creating more sophisticated and versatile laparoscopic tools.

The present invention is directed to a multifunctional laparoscopic instrument designed to enhance intraoperative efficiency by consolidating several operative capabilities into a single handheld device. In one embodiment, the instrument includes a reusable hilt with an integrated joystick, trigger and mode selection interface, configured to control an interchangeable shaft with an articulated distal end. The shaft may incorporate internal channels for suction, irrigation, and electrosurgical energy delivery, thereby minimizing the need for instrument exchanges during minimally invasive procedures.

The device is operable in multiple modes—including standard grasping, suction, irrigation, and electrosurgery—which may be selected via ergonomically placed buttons on the hilt. An LED indicator on the device may provide visual feedback corresponding to the current operational mode. The joystick may be configured to enable multi-axis control of the distal end, such as pitch and yaw articulation, while also supporting axial depression to activate electrosurgical functions. Additional features may include bidirectional or analog trigger control for proportional actuation of distal end opening and closing, as well as lateral bidirectional control for axial rotation of the shaft.

In certain embodiments, the instrument includes internal logic circuitry—such as a microcontroller or microprocessor—configured to coordinate the functional subsystems of the device. These subsystems may include motor-driven articulation, fluidic valves, haptic feedback, controlling flow of current for electrosurgery, and sensor integration. The device architecture allows for the use of contactless sensors (e.g., Hall effect) in the trigger and joystick to enhance precision and sterility.

Furthermore, the current device includes the integration of multiple functionalities into a single laparoscopic instrument and that integration presents several engineering challenges. These include miniaturization of components, electrical isolation for safety, fluid management within a confined space, and maintaining precise control over articulation and grasping mechanisms. Additionally, ensuring sterility and durability of multi-function instruments can be complex, particularly when combining electronic and fluidic systems.

According to an aspect of the present disclosure, a laparoscopic instrument is provided. The laparoscopic instrument includes a handle assembly, an elongate shaft extending distally from the handle assembly, and an end effector assembly disposed at a distal end of the elongate shaft. The handle assembly includes a housing, a trigger movably coupled to the housing, and a joystick movably coupled to the housing. The handle assembly further includes a mode selection interface disposed on an exterior surface of the housing and configured to enable selection between a plurality of operative modes. The handle assembly also includes a microcontroller unit (MCU) disposed within the housing and operably coupled to the trigger, the joystick, and the mode selection interface. The MCU is configured to receive input signals from the trigger, the joystick, and the mode selection interface, and to control operation of the laparoscopic instrument based on the received input signals and a selected operative mode.

According to other aspects of the present disclosure, the laparoscopic instrument may include one or more of the following features. The trigger may be configured to detect user-applied displacement in inward, outward, and lateral directions. The joystick may be configured to detect user-applied displacement in pitch, yaw, and depression directions. The mode selection interface may include a plurality of buttons, each button corresponding to a distinct operative mode. The plurality of operative modes may include a normal mode, a suction mode, an irrigation mode, and an electrosurgical mode. The MCU may be configured to dynamically reassign functions of the trigger and the joystick based on the selected operative mode.

The laparoscopic instrument may further include at least one Hall effect sensors operably coupled to the trigger and configured to detect displacement of the trigger. The laparoscopic instrument may also include at least one Hall effect sensors operably coupled to the joystick and configured to detect displacement of the joystick. The end effector assembly may include a pair of jaw members movable between an open position and a closed position, along with an intermediate member that holds the jaws. The intermediate member allows for movement of the jaw member as a unit in the pitch axis. The elongate shaft may include at least one fluid channel extending therethrough.

According to another aspect of the present disclosure, the laparoscopic instrument may include a power supply unit disposed outside of or within the housing and operably coupled to the MCU. The power supply unit may be configured to receive AC power and convert the AC power to DC power for use by components of the laparoscopic instrument. The laparoscopic instrument may also include a plurality of motors disposed within the housing and operably coupled to the MCU. The plurality of motors may be configured to control movement of the end effector assembly based on input signals received from the trigger and the joystick.

According to other aspects of the present disclosure, the laparoscopic instrument may include a gearbox assembly removably coupled to the handle assembly. The gearbox assembly may include a plurality of pulleys, which is operably coupled to the plurality of motors and configured to transmit motion to the end effector assembly. The gearbox assembly may further include at least one fluid coupling interface configured to establish fluid communication between the at least one fluid channel and a fluid source or sink. The gearbox assembly may also include an electrical connector configured to establish the path that current will flow through for electrosurgery, and the end effector assembly.

The laparoscopic instrument may include a split-shell housing configured to allow access to internal components of the handle assembly. The split-shell housing may include two or more interlocking segments that can be separated for maintenance or component replacement. The laparoscopic instrument may further include at least one solenoid valve operably coupled to the MCU and configured to control fluid flow through the at least one fluid channel based on the selected operative mode and input signals received from the trigger.

According to a preferred embodiment of the invention the present invention relates to laparoscopic instrument s for minimally invasive procedures, and more specifically to a multifunctional laparoscopic tool that consolidates multiple operative functions—including tissue grasping, fluid suction and irrigation, distal tip articulation, and electrosurgical energy delivery (electrocautery) —into a single hand-operated instrument. The invention enables real-time control of these modalities through an integrated user interface, eliminating the need for frequent instrument exchanges during surgery. This integration is achieved through a compact internal design that coordinates electronic actuation, control logic, and fluid routing within a modular, sterilizable form factor. The invention provides a practical alternative to current multi-instrument workflows and robotic systems by combining responsive control, multifunctional capability, and ergonomic efficiency in a standalone device. Importantly, the invention is not a mere aggregation of existing functions, but a novel integration that enables coordinated, real-time control of multiple surgical modalities through a unified interface and embedded actuation system—overcoming key limitations of both conventional handheld tools and high-cost robotic platforms. The internal coordination of motorized articulation, fluid management, and energy delivery within a spatially constrained, sterilizable handheld form factor addresses longstanding engineering challenges in multifunctional laparoscopic instrument design.

Still other objects and advantages of the invention will in part be obvious and will in part be apparent from the specification.

The above and other objects, which will be apparent to those skilled in the art, are achieved in the present invention, which is directed to a laparoscopic instrument, comprising:
a. a handle assembly including a housing, a trigger movably coupled to the housing, a joystick movably coupled to the housing, a mode selection interface disposed on an exterior surface of the housing, and a microcontroller unit (MCU) disposed within the housing;
b. an elongate shaft extending distally from the handle assembly; and
c. an end effector assembly disposed at a distal end of the elongate shaft, wherein the MCU is operably coupled to the trigger, the joystick, and the mode selection interface, and is configured to receive input signals from the trigger, the joystick, and the mode selection interface, and to control operation of the laparoscopic instrument based on the received input signals and a selected operative mode.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a system architecture overview of a computing device, according to aspects of the present disclosure.

FIG. 2 illustrates an orthogonal side view of a laparoscopic instrument, according to an embodiment.

FIG. 3 illustrates a browser menu interface for managing a laparoscopic instrument assembly, according to aspects of the present disclosure.

FIG. 4 illustrates an orthogonal view of the laparoscopic instrument of FIG. 2, according to an embodiment.

FIG. 5 illustrates an orthogonal view of a trigger assembly mechanism, according to aspects of the present disclosure.

FIG. 6A illustrates an orthogonal view of a joystick control assembly, according to an embodiment.

FIG. 6B illustrates an orthogonal view of the joystick control assembly of FIG. 6A, according to aspects of the present disclosure.

FIG. 7 illustrates a section view of a gearbox housing from an instrument side, according to an embodiment.

FIG. 8 illustrates a section view of a gearbox housing showing internal components, according to aspects of the present disclosure.

FIG. 9 illustrates a view of the fluid transport system of a laparoscopic instrument, according to an embodiment.

FIG. 10 illustrates a section view of a laparoscopic instrument assembly, according to aspects of the present disclosure.

FIG. 11 illustrates a section view of a portion of the laparoscopic instrument, according to an embodiment.

FIG. 12 illustrates a section view of a gearbox housing showing internal components, according to aspects of the present disclosure.

FIG. 13 is a view of the distal assembly of a laparoscopic instrument, according to an embodiment.

FIG. 14 is a view of the distal assembly of FIG. 13, according to aspects of the present disclosure.

FIG. 15 is a view of a portion of the laparoscopic instrument showing control cables, according to an embodiment.

FIG. 16 illustrates a flowchart showing a process for operating a laparoscopic instrument, according to aspects of the present disclosure.

FIG. 17 illustrates a flowchart of an electrocautery control process, according to an embodiment.

FIG. 18 illustrates a flowchart showing a motor-pulley coupling interface operation process, according to aspects of the present disclosure.

FIG. 19 illustrates a flowchart of a microcontroller unit control process, according to an embodiment.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Prior to a discussion of the preferred embodiment of the invention, it should be understood that while the features and advantages of the invention are illustrated in terms of a laparoscopic instrument for use in laparoscopic surgery.

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a laparoscopic instrument for use in minimally invasive procedures. In some cases, the laparoscopic instrument may incorporate multiple functionalities into a single handheld device. The laparoscopic instrument may include components for mechanical manipulation, fluid management, and energy delivery during surgical procedures.

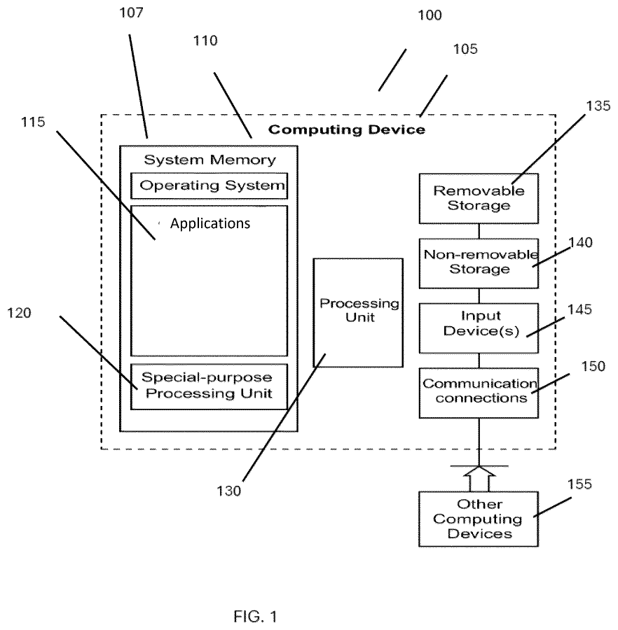

FIG. 1 illustrates a system architecture overview 100 of a computing device 105 that may be used in conjunction with the laparoscopic instrument. The computing device 105 may include a system memory 107 containing an operating system 110 and applications 115. A special purpose processor 120 may be included for specialized computing tasks related to the laparoscopic instrument's operation. The computing device 105 may also incorporate a processing unit 130 for handling computational tasks. For data storage, the computing device 105 may include both removable storage 135 and nonremovable storage 140. An input device 145 may allow for user interaction with the computing device 105. Communication connections 150 may enable the computing device 105 to interface with external other computing devices 155.

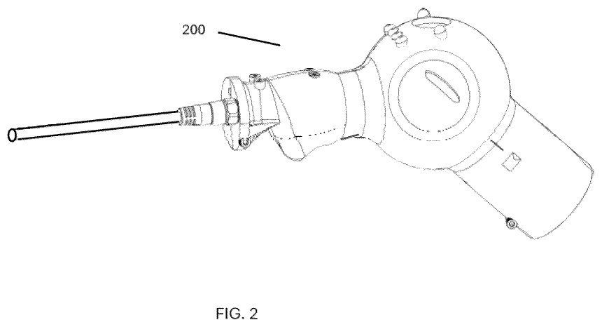

FIG. 2 shows an orthogonal side view of a laparoscopic instrument 200. The laparoscopic instrument 200 may comprise an elongated body with a shaft extending from the body. This configuration may allow for insertion through surgical access ports during minimally invasive procedures.

The laparoscopic instrument 200 may integrate various subsystems to enable multiple functions. These subsystems may include mechanical actuation components, fluid delivery and removal pathways, and energy delivery mechanisms. The integration of these functionalities into a single device may reduce the need for instrument exchanges during surgical procedures.

In some cases, the laparoscopic instrument 200 may be designed for single-handed operation, allowing a surgeon to control multiple functions without changing hand position. The instrument may incorporate user interface elements for mode selection and control of various functions.

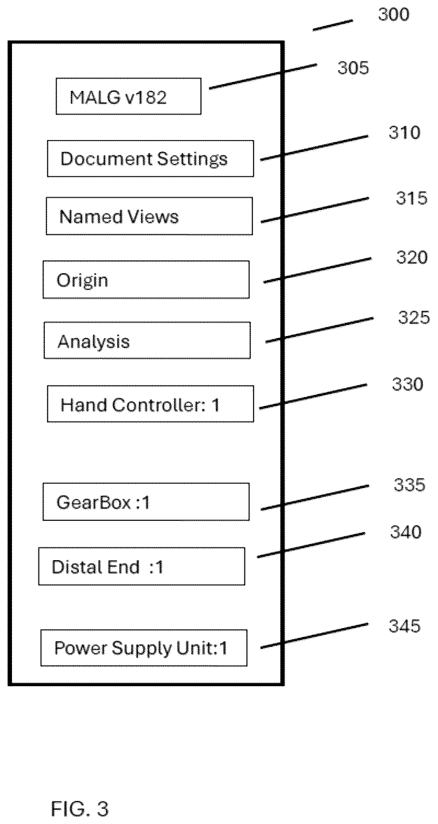

FIG. 3 illustrates a browser menu 300 that may be used to manage the components and settings of the laparoscopic instrument assembly. This interface may provide access to various subsystems and configuration options for the device.

The laparoscopic instrument 200 may be designed to interface with external systems for power, fluid supply, and control. In some cases, the instrument may incorporate modular components to allow for customization or replacement of specific parts.

The system architecture overview 100 illustrates the components and structure of a computing device 105 that may be used to control and operate the laparoscopic instrument 200. The computing device 105 may include a system memory 107 that contains an operating system 110 and applications 115 for managing various functions of the laparoscopic instrument 200.

In some cases, the computing device 105 may incorporate a special purpose processor 120 designed to manage specialized computing tasks related to the operation of the laparoscopic instrument 200. The computing device 105 may also include a processing unit 130 for executing general computational tasks and coordinating the functions of various components within the laparoscopic instrument 200.

The computing device 105 may be equipped with both removable storage 135 and nonremovable storage 140 to store data related to surgical procedures, instrument configurations, and operational parameters. An input device 145 may be provided to allow user interaction with the computing device 105, enabling surgeons or medical staff to input commands or adjust settings for the laparoscopic instrument 200.

The computing device 105 may include communication connections 150 that enable connectivity with external computing devices 155. These communication connections 150 may facilitate data exchange, remote monitoring, or software updates for the laparoscopic instrument 200.

In some cases, the laparoscopic instrument 200 may incorporate a switching power supply to convert alternating current (AC) from a standard electrical outlet to direct current (DC) required by the instrument's internal components. This power conversion may ensure stable and appropriate power delivery to various subsystems within the laparoscopic instrument 200.

The laparoscopic instrument 200 may include a microcontroller unit (MCU) to coordinate inputs and outputs. This MCU may process signals from sensors, user inputs, and other components, and generate appropriate control signals for actuators, motors, and other functional elements within the laparoscopic instrument 200. The MCU may interface with the computing device 105 through the communication connections 150, allowing for real-time control and data exchange.

The browser menu 300 displayed on the computing device 105 may provide a user interface for accessing and managing various aspects of the laparoscopic instrument 200. This interface may allow users to configure settings, monitor instrument status, and control different functions of the laparoscopic instrument 200 through interaction with the computing device 105.

The laparoscopic instrument 200 may comprise an elongated body with a shaft extending from the body, as shown in FIG. 2. This configuration may allow for insertion through surgical access ports during minimally invasive procedures. In some cases, the laparoscopic instrument 200 may incorporate a modular split-shell housing to facilitate access to internal components. The housing may be fabricated from sterilization-compatible materials such as PEEK or PPSU.

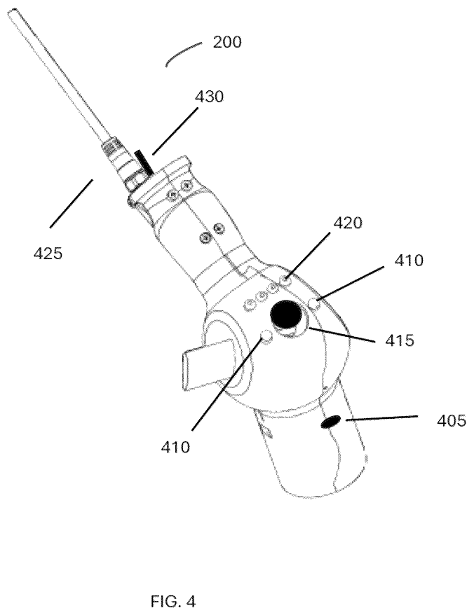

FIG. 4 illustrates an orthogonal view of the laparoscopic instrument 200, revealing several key components. A joystick controller 415 may be positioned on the upper surface of the laparoscopic instrument 200 for user input. Adjacent to the joystick controller 415, the laparoscopic instrument 200 may include multiple mode selectors 420. In some cases, four mode selection buttons may be incorporated, corresponding to Normal, Suction, Irrigation, and Electrosurgery modes. The laparoscopic instrument 200 may also feature rotation controls 410 positioned on the lateral sides of the device body.

An indicator light 405 may be integrated into the housing of the laparoscopic instrument 200 to provide visual feedback of the instrument's operational status. The laparoscopic instrument 200 may include an electrosurgery connector 425 extending from the proximal end for connection to external power sources. A barbed fitting 430 may also be located at the proximal end of the laparoscopic instrument 200 to facilitate fluid connections.

The components of the laparoscopic instrument 200 may be arranged in an ergonomic configuration that allows for single-handed operation. The joystick controller 415 and mode selectors 420 may be positioned for thumb access, while the rotation controls 410 may be accessed without changing the grip position.

As previously disclosed FIG. 3 shows a browser menu 300 that may be used to manage the components and settings of the laparoscopic instrument 200. The browser menu 300 may include a device name field 305, a document settings field 310, and a named views field 315 for accessing different perspectives of the assembly. An origin field 320 and an analysis field 325 may provide access to reference coordinates and analytical functions, respectively.

The browser menu 300 may also include a hand controller field 330 for accessing controller-related settings, a gearbox field 335 for managing gearbox components, and a distal end field 340 for accessing settings related to the distal portion of the instrument. A power supply field 345 may allow configuration of power supply parameters for the laparoscopic instrument 200.

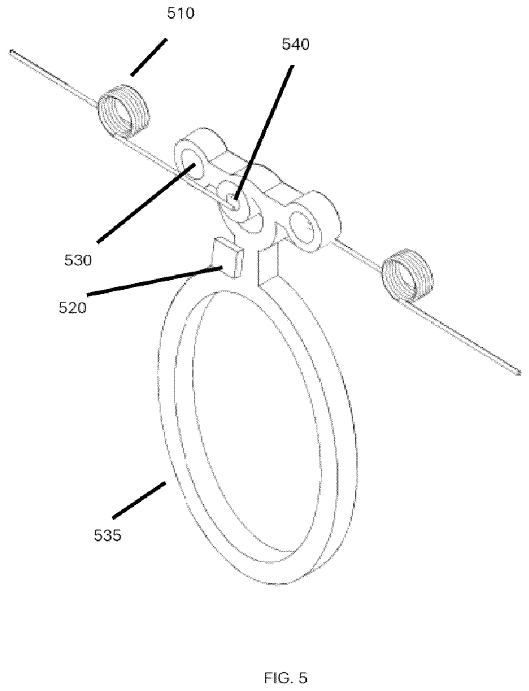

The laparoscopic instrument 200 may incorporate a trigger assembly that enables precise control of various instrument functions. FIG. 5 illustrates an orthogonal view of the trigger assembly mechanism, revealing its internal components and structure.

In some cases, the trigger assembly may include a trigger 535 that serves as the primary user input for controlling grasper actuation, fluid flow, or other functions depending on the selected operational mode. The trigger 535 may be mechanically coupled with two torsional springs 510 positioned on opposite sides of the trigger 535. These torsional springs 510 may provide resistance and return force for the trigger 535 movement, offering tactile feedback to the user during operation.

The trigger assembly may incorporate a torsional spring lock 530 that secures the torsional springs 510 in place. This torsional spring lock 530 may help maintain proper alignment and tension of the torsional springs 510 within the assembly, ensuring consistent performance over repeated use cycles.

In some cases, the trigger assembly may include a hall effect sensor 520 positioned adjacent to the trigger 535. The hall effect sensor 520 may be configured to detect the movement and position of the trigger 535 without physical contact. This non-contact sensing method may allow for precise measurement of trigger displacement while minimizing mechanical wear.

The trigger assembly may also incorporate a bearing 540 that facilitates smooth rotational movement of the trigger mechanism. The bearing 540 may support the mechanical components while reducing friction during trigger actuation, contributing to the overall responsiveness and durability of the assembly.

The hall effect sensor 520 may generate electrical signals corresponding to the trigger's displacement. These signals may be transmitted to the microcontroller unit (MCU) housed within the laparoscopic instrument 200. The MCU may process these signals to determine the degree of trigger actuation and translate this input into appropriate control commands for various instrument functions.

In some cases, the trigger assembly may be designed to detect user-applied displacement in both inward (compressive) and outward (releasing) directions. This bidirectional sensing capability may enable more nuanced control over instrument functions, such as proportional control of grasper closure or fluid flow rates.

The trigger assembly may be configured to operate in different modes based on the selected function of the laparoscopic instrument 200. For example, in a normal grasping mode, trigger actuation may control the opening and closing of the distal grasper. In suction or irrigation modes, the trigger may instead regulate fluid flow through the instrument.

The mechanical design of the trigger assembly, combined with the precision sensing capabilities of the hall effect sensor 520, may allow for fine control over the laparoscopic instrument's functions. This integration of mechanical and electronic components may contribute to the overall versatility and usability of the laparoscopic instrument 200 in various surgical procedures.

The laparoscopic instrument 200 may incorporate a joystick control system to enable multi-axis control of the instrument's distal end and facilitate mode selection.

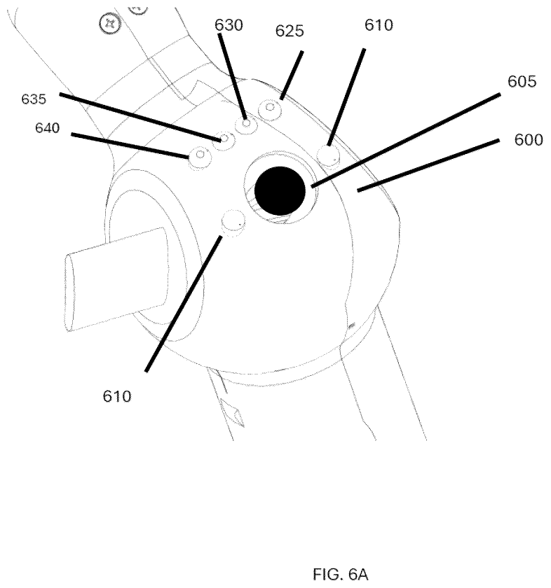

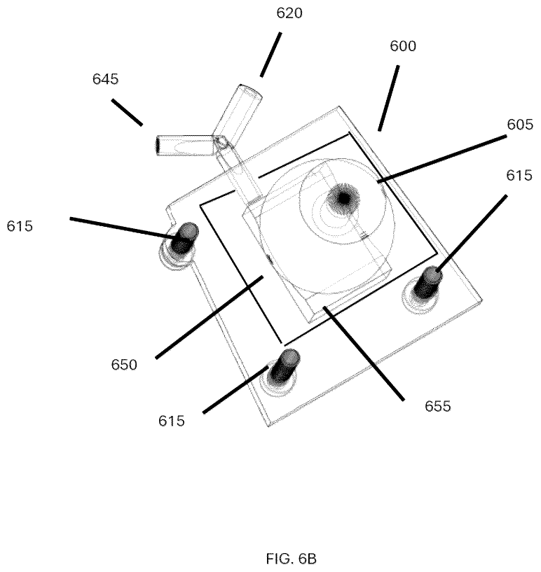

FIG. 6A illustrates a view of a joystick control assembly 600, revealing the arrangement of control buttons positioned around a central joystick 605.

In some cases, the joystick control assembly 600 may include a roll control button 610 positioned on one side of the joystick 605. The joystick control assembly 600 may incorporate several mode selection buttons arranged around the joystick 605, including an irrigation control button 625, a suction control button 630, a normal operation button 635, and an electrosurgery button 640. These buttons may be arranged in a configuration that allows for ergonomic access during operation of the laparoscopic instrument 200.

The joystick 605 may be centrally positioned within the joystick control assembly 600 to enable control of the distal end articulation. In some cases, the joystick 605 may utilize Hall effect sensors to detect displacement. This non-contact sensing method may allow for precise measurement of joystick movement while minimizing mechanical wear.

FIG. 6B illustrates an orthogonal view of the joystick control assembly 600, revealing additional components. The joystick control assembly 600 may include multiple mounting screws 615 arranged around the perimeter to secure components in place. A motor output 620 may be provided for transmitting power to drive components within the laparoscopic instrument 200.

The joystick control assembly 600 may also include a power supply connection 645 for receiving electrical power. In some cases, a position sensor 655 may be incorporated to detect the movement and position of the joystick 605 during operation. The position sensor 655 may work in conjunction with the Hall effect sensors to provide accurate and responsive control inputs.

The components of the joystick control assembly 600 may be arranged in a compact configuration that allows for efficient packaging while maintaining accessibility. The joystick 605 may be positioned to enable user control, while the position sensor 655 monitors its movement. The mounting screws 615 may be strategically placed to provide secure attachment points.

In some cases, the joystick control assembly 600 may interface with the microcontroller unit (MCU) housed within the laparoscopic instrument 200. The MCU may process signals from the joystick 605 and associated sensors to determine the degree and direction of joystick actuation. These inputs may be translated into appropriate control commands for various instrument functions, such as distal end articulation or mode selection.

The joystick control system may be designed to operate in different modes based on the selected function of the laparoscopic instrument 200. For example, in a normal operation mode, joystick actuation may control the articulation of the distal end. In electrosurgery mode, the joystick may be used to adjust energy delivery parameters.

The integration of the joystick control system with the laparoscopic instrument 200 may allow for precise and intuitive control over multiple instrument functions. This combination of mechanical design and electronic sensing may contribute to the overall versatility and usability of the laparoscopic instrument 200 in various surgical procedures.

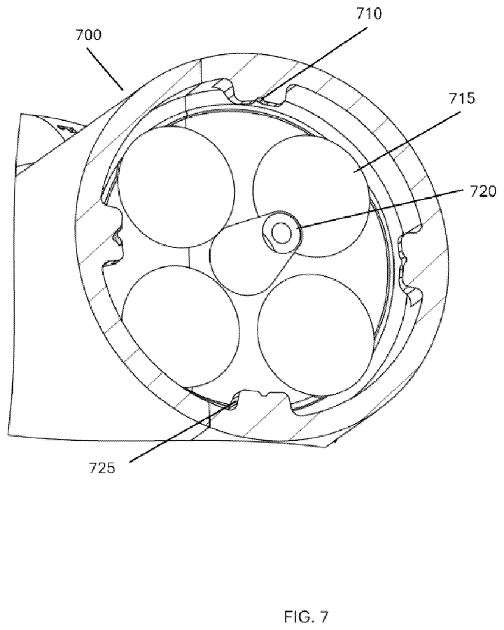

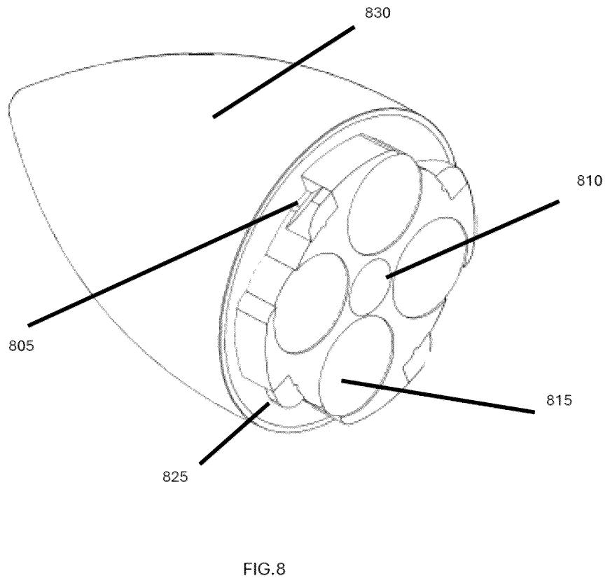

The laparoscopic instrument 200 may incorporate a gearbox assembly to facilitate mechanical power transmission and control of the instrument's distal end. FIG. 7 illustrates a section view of a first gearbox housing 700 from the instrument side of the laparoscopic instrument 200, revealing several key components and interfaces. The gear box has two parts a first gear box housing 700 shown in FIG. 7 and a second gearbox housing 830 shown in FIG. 8.

In some cases, the first gearbox housing 700 may include electrosurgical contacts 710 positioned in the upper portion to enable electrical connectivity for energy delivery functions. The first gearbox housing 700 may also incorporate multiple magnetic shaft couplers 715 arranged within the housing to provide mechanical coupling capabilities between the gearbox and other components of the laparoscopic instrument 200.

The first gearbox housing 700 may feature a fluid outlet port 720 integrated into the structure to allow fluid transfer through the instrument. This fluid outlet port 720 may connect to internal fluid channels within the laparoscopic instrument 200, enabling irrigation or suction functions depending on the selected operational mode.

In some cases, the first gearbox housing 700 may include twist lock tabs 725 that enable secure mechanical attachment to mating components within the laparoscopic instrument 200. These twist lock tabs 725 may provide a quick-connect mechanism for assembling or disassembling the gearbox from the main body of the instrument.

The gearbox assembly may utilize a pulley and cable system for motion transmission to the distal end of the laparoscopic instrument 200. This system may allow for precise control over the movement and articulation of the instrument's end effector.

In some cases, the laparoscopic instrument 200 may incorporate four stepper motors to control various degrees of freedom at the distal end. These motors may be responsible for actuating grasper movement, pitch articulation, yaw articulation, and roll rotation. The gearbox assembly may serve as an interface between these motors and the mechanical components at the distal end of the instrument.

The first gearbox housing 700 may be designed to interface with the motor outputs from the main body of the laparoscopic instrument 200. In some cases, the magnetic shaft couplers 715 may facilitate the connection between the motor shafts and the corresponding pulleys within the gearbox assembly.

FIG. 8 illustrates another view of a second gearbox housing 830, revealing additional components and interfaces. The second gearbox housing 830 may include electrosurgical contacts 805 positioned at the proximal end for electrical connectivity with the main body of the laparoscopic instrument 200.

In some cases, the second gearbox housing 830 may incorporate a fluid interface 810 to provide a watertight seal for fluid transfer between the main body and the gearbox assembly. This fluid interface 810 may connect with the fluid outlet port 720 to maintain a continuous fluid pathway through the instrument.

The second gearbox housing 830 may feature magnetic couplers 815 that enable secure mechanical connection with corresponding components in the main body of the laparoscopic instrument 200. These magnetic couplers 815 may provide quick and reliable engagement while allowing for easy disassembly when needed.

Around the perimeter of the gearbox housing 830, there may be locking slots 825 that facilitate mechanical attachment and alignment with mating components. These locking slots 825 may correspond to the twist lock tabs 725 on the opposing interface, ensuring proper orientation and secure connection between the gearbox and the main body of the laparoscopic instrument 200.

The gearbox assembly may serve as a crucial interface between the electronic control systems, such as the microcontroller unit (MCU) and motor drivers, and the mechanical components responsible for distal end articulation and actuation. By housing the pulley and cable system, the gearbox may enable the translation of motor rotation into precise movements of the laparoscopic instrument's end effector.

In an alternative embodiment the gearbox assembly is designed as a modular component that can be easily separated from the main body of the laparoscopic instrument 200. This modular design may facilitate maintenance, cleaning, and sterilization of the instrument between surgical procedures.

In accordance with one embodiment of the present invention, the laparoscopic instrument is configured with a modular split-shell housing, wherein the outer body of the device is divided along a central longitudinal or transverse plane, thereby forming two mating halves or shell segments. This configuration facilitates enhanced access to internal components, particularly those associated with fluid transport and electromechanical routing, and is specifically designed to aid in sterilization and routine maintenance.

The split-shell housing is secured by fasteners, latching clips, or interlocking tabs configured to permit non-destructive disassembly by qualified personnel. Upon separation of the shell halves, the internal lumen—comprising one or more flexible fluid transport tubes—is rendered accessible, allowing for visual inspection, cleaning, and, where necessary, replacement of the tubing or associated valves and seals.

This housing architecture offers several advantages, including:

Improved Sterility Assurance: By enabling full or near-full exposure of the internal fluid pathways and drive mechanisms, the device permits more thorough decontamination during reprocessing cycles, thus mitigating the risk of residual biofluid or microbial accumulation in inaccessible regions.

Component Replacement Efficiency: The split-shell design allows modular replacement of internal tubing, which may degrade due to repeated use, fluid exposure, or sterilization cycles. The tubing may be connected using snap-fit connectors, luer-lock fittings, or compression seals, allowing tool-free detachment and replacement.

Serviceability and Longevity: Maintenance procedures, such as tubing replacement or inspection of wiring and connectors, can be performed without damaging the primary housing or voiding device integrity, thereby extending the operational lifespan of the reusable components.

In certain embodiments, the split-shell interface includes alignment pins, gasketed seals, or dowel-and-slot systems to maintain mechanical integrity, prevent fluid or debris ingress during use, and ensure precise reassembly post-sterilization. The materials used for the housing may be selected from biocompatible, high-temperature-resistant polymers (e.g., PEEK, PPSU) suitable for repeated autoclave or hydrogen peroxide plasma sterilization cycles.

This design thereby promotes safe reusability, modular servicing, and regulatory compliance with established sterilization standards in surgical environments.

In one preferred embodiment of the invention, the laparoscopic instrument features a modular split-shell housing composed of two or more interlocking outer segments that enclose the internal electromechanical components of the device. These segments are mechanically joined along a central longitudinal seam (or alternatively, along a transverse plane), forming a clam-shell style enclosure that is both field-serviceable and sterilization-compatible.

The split-shell architecture permits repeatable, tool-assisted disassembly by trained surgical technicians or authorized personnel without compromising the long-term structural integrity or alignment of the instrument. The enclosure may be secured using one or more of the following mechanisms:
a. Medical-grade snap-fit interlocks
b. Threaded fasteners with sterilizable heads (e.g., Torx, hex)
c. Compression-fit clips or quarter-turn bayonet mounts

To ensure precise reassembly, the mating interfaces may include integrated alignment features such as dowels, guide tracks, or keyed slots. These help maintain proper positioning of the internal components and ensure alignment of the distal articulation mechanisms, circuit interfaces, and fluid channels across high-cycle clinical use.

A medical-grade elastomeric gasket may be seated circumferentially along the shell interface, ensuring:
a. Watertight sealing of internal electronics and mechanical assemblies,
b. Barrier protection against biological fluid ingress,
c. Vibration dampening between housing halves during repetitive instrument actuation.

The laparoscopic instrument is designed to provide an internal layout and a biological interface management system which can be appreciated upon opening the internal compartment of the laparoscopic instrument which comprises:
a. A sealed fluid transport lumen, preferably composed of flexible, medical-grade plastic tubing (e.g., PTFE, silicone)
b. Dedicated motor compartments and/or control PCBs
c. Embedded sensor arrays, including Hall effect sensors and displacement encoders
d. Internal routing for electrosurgical cabling, joystick signal lines, and trigger-actuation circuitry.

Critically, the plastic fluid tubing is the only internal component of the hilt that directly interfaces with biological specimens or patient-derived fluids. Its modular mounting system—consisting of non-permanent clip channels, quick-release brackets, or snap-in grommets—allows the tubing to be removed and replaced independently of the rest of the system. This enables cost-effective reuse of the housing and electronics while maintaining full biological safety compliance.

Other components within the housing are mechanically or magnetically mounted to withstand repeated sterilization cycles (≥134° C.) and maintain long-term device integrity.

### Materials and Exterior Design

The shell halves are fabricated from high-performance, sterilization-stable polymers such as PEEK, PPSU, or Ultem, chosen for their:
a. High mechanical strength-to-weight ratios
b. Autoclave and chemical sterilant resistance
c. Low moisture absorption
d. Biocompatibility and clinical durability

Externally, the shell is contoured for ergonomic handling and shaped to support intuitive alignment of the surgeon's thumb and index finger with the joystick and trigger, respectively. Textured surfaces, grip ridges, or visual etchings may assist with hand positioning and orientation during procedures.

### Workflow: Use of the Split-Shell Housing in Clinical Practice

### Initial Use

Prior to first use, the fully assembled device undergoes standard sterilization without the tubing in place. After sterilization, a sterile plastic tubing segment is opened from its packaging. The technician then opens the device, inserts the tubing into its designated channel within the hilt, securely closes the housing, and prepares the instrument for immediate use in the operating room.

### Intraoperative Application

During surgery, the tubing comes into direct contact with biological material (e.g., blood, irrigation fluid, debris) via suction or irrigation. The plastic tubing begins at the interface between the shaft and the hilt, and is the only internal component within the hilt that is exposed to biological fluids.

### Postoperative Decontamination

After use, the instrument is returned to sterile processing. A technician separates the shaft from the hilt. He then removes a small number of fasteners (or unlatches the interlock system) to separate the split-shell housing. The tubing is removed and discarded.

### Component Inspection and Service

With the housing open and tubing removed, the technician can inspect the internal components, including the fluid lumen, motors, sensor assemblies, circuit wiring, and firmware ports. If necessary, they may recalibrate sensors, clear obstructions from channels, or perform software updates. If all components are found to be in proper working order, the hilt is reassembled and sent for sterilization with the shaft.

### Reassembly and Sterilization

Following sterilization, and at the beginning of a new case, a new sterile tubing segment is opened from its packaging. The technician reopens the device, inserts the tubing into the designated channel within the hilt. The housing is then securely closed, tested for seal integrity, and ready for use.

The laparoscopic instrument inner body components comprise of:

### Power System

a. The present invention includes a multi-stage power distribution architecture configured to supply regulated electrical power to various subsystems of the laparoscopic instrument, including actuators, motors, sensors, logic circuits, and electrosurgical energy delivery components. The power supply unit will be a separate entity connected to the hand controller through a multiplexed wire, which allows for different voltages to be transmitted through one wire.
   i. 1. Primary Power Input
      1. The system receives primary input power from a wall outlet, operable with either 110V or 220V alternating current (AC), depending on geographic and clinical requirements. This input is directed to a switching power supply unit (PSU), which is responsible for converting high-voltage AC to regulated low-voltage direct current (DC) outputs suitable for downstream components.
   ii. 2. Low-Voltage DC Conversion
      1. The switching PSU generates a 12V DC rail, which serves as the main low-voltage power bus for the instrument's internal electronics and actuators. From this 12V DC rail, multiple regulated voltage branches are derived to support specific system components:
      2. The 12V DC rail provides power to electric motors and solenoid valves, which are responsible for mechanical operations, including grasper control, articulation, control of fluid transport and cable-driven mechanisms.
      3. A second converter outputs 5V at 2A, supplying power to the digital logic subsystem and sensor array, including embedded Hall effect sensors, mode selectors, displacement encoders, and feedback circuits.

All subsystems share a common ground reference and may be isolated or filtered to mitigate electrical noise and crosstalk, particularly in environments involving simultaneous analog and digital signal processing.

The Micro controller unit controls the electric motors.
iii. 3. High-Voltage Isolation for Electrosurgery
   1. In the primary embodiment, the laparoscopic instrument is configured to interface with a standalone electrosurgical generator unit, commonly present in modern operating theaters. The instrument includes an external high-voltage input port designed to accept industry-standard electrosurgical connectors, such as monopolar pencil jacks or bipolar output leads. This interface enables the device to utilize regulated high-voltage current supplied by the external electrosurgical generator for the purpose of tissue cauterization, coagulation, or cutting, depending on the surgical context.
   2. Electrical activation of the external current source is governed by the instrument's internal logic subsystem, which may include user-initiated controls such as joystick depressions, mode-specific triggers, or capacitive switches. The system may incorporate electrical or optical isolation elements (e.g., optocouplers, isolated gate drivers, or solid-state relays) to safely modulate and gate the delivery of electrosurgical energy, thereby maintaining galvanic separation between the control circuitry and the external high-voltage source.

This configuration allows the laparoscopic instrument to:
a. Leverage medically certified high-voltage power supplies already in use in the surgical environment;
b. Reduce thermal and spatial constraints within the device body;
c. Simplify compliance with regulatory standards related to high-voltage safety and EMI.
   iv. Power Regulation and Control Logic
      1. The system further comprises real-time monitoring and regulation circuitry, integrated within the logic subsystem, to ensure stable voltage delivery, overcurrent protection, and operational diagnostics. Voltage and current sensors may be used to monitor power rails and provide feedback to the microcontroller for safety shutdown or performance optimization.

The laparoscopic instrument includes an integrated fluid transport unit configured to selectively regulate the flow of irrigation and suction media through the laparoscopic instrument during minimally invasive procedures. This subsystem is operable in conjunction with external fluid sources, and is controlled through internal logic based on user-selected operating modes.
a. Valve Configuration
   i. The fluid transport unit comprises at least two electronically actuated solenoid valves, each corresponding to one of the fluid functions: irrigation and suction, respectively. Each solenoid valve is positioned in-line with a dedicated fluid channel and is paired with a corresponding check valve or one-way valve, which serves to prevent backflow, thereby ensuring unidirectional, contamination-free operation of both fluid inflow and outflow paths.
   ii. The solenoid valves are driven by the internal low-voltage control electronics and may incorporate fail-safe default states (e.g., normally closed) to ensure patient and operator safety in the event of power loss or system fault.
b. External Source Interface
   i. The irrigation and suction lines are designed to interface with standard hospital infrastructure, such as external saline or sterile water reservoirs (for irrigation) and central or portable vacuum systems (for suction). This is achieved through industry-standard luer-lock or barbed hose fittings, enabling seamless integration with existing surgical tubing networks. The fittings are constructed from biocompatible, sterilizable materials to preserve fluid path sterility and device reusability.
c. Mode-Dependent Activation
   i. Fluid flow is governed by the mode selection system described in prior sections. When the device is placed into Suction Mode or Irrigation Mode—either via button selection or automated control logic—the actuation of the primary trigger mechanism results in opening of the corresponding solenoid valve, allowing for immediate flow of the selected fluid.
   ii. In one embodiment, trigger depression depth or force may be analog-monitored (e.g., via Hall effect or pressure sensors) to modulate the rate of fluid flow, providing user-variable control over suction or irrigation strength.
d. Control Integration and Safety
   i. All valve control signals are routed through the Microcontroller Unit (MCU), which ensures that:
      1. Only one fluid path is active at a time;
      2. Fluid delivery is synchronized with user intent;
      3. System states (e.g., valve open/closed, flow detected) are logged or monitored.

Optional flow sensors or pressure transducers may be included in line to provide closed-loop monitoring, automatic cutoff, or feedback to the operator via haptic or visual indicators.

The laparoscopic instrument comprises a modular micromotor subsystem configured to drive the mechanical articulation and distal actuation functions of the instrument. In the preferred configuration, the system utilizes four stepper motors, housed within the main body of the device, to control the following degrees of freedom:
a. Opening and closing of the grasper,
b. Pitch articulation,
c. Yaw articulation, and
d. Axial roll rotation of the distal end.

Each stepper motor is paired with a dedicated motor driver circuit, which receives power from the internal switching power supply unit (PSU) and command signals from the Microcontroller Unit (MCU). The driver circuits are physically co-located with their respective motors within the main housing to minimize signal degradation, reduce electromagnetic interference (EMI), and optimize thermal routing.

### Power and Cooling Architecture

a. All four stepper motors receive regulated DC power, typically in the range of 7.4V to 12V, derived from a common PSU rail. Each motor driver circuit includes integrated features such as:
   i. Current limiting,
   ii. Microstepping capability (e.g., 1/16 or 1/32),
   iii. Overtemperature protection, and
   iv. Flyback suppression circuitry.

To ensure thermal stability in the compact and sealed surgical housing, passive thermal management is employed. Each driver and motor module is fitted with low-profile aluminum or copper heatsinks, adhered using thermally conductive adhesive or tape. The housing itself may incorporate heat-conductive inserts or thermal vias to dissipate heat during prolonged use.

### Safety and Fault Detection Protocols

a. To maintain operational safety and system reliability, the instrument includes multi-tiered fault detection and shutdown mechanisms, such as
   i. Current sensors on each driver channel to detect overcurrent conditions or motor stalls,
   ii. NTC thermistors or embedded IC temperature sensors to monitor motor heating,
   iii. Watchdog timers within the MCU to detect firmware execution anomalies,
   iv. Operational state monitoring, such as trigger input vs. motor response correlation, to detect cable breakage or actuation failure.

Upon detection of a fault condition, the MCU is configured to immediately disable all motor outputs and energy delivery systems, and may alert the operator through haptic, auditory, or visual cues.

### Closed-Loop Stepper Control

a. In one embodiment, the system employs a closed-loop motor control architecture. Each stepper motor is coupled with a rotary encoder (e.g., magnetic or optical) mounted on the motor shaft or pulley output. Encoder feedback is continuously monitored by the MCU to:
   i. Confirm actual position matches commanded position,
   ii. Detect and compensate for missed steps,
   iii. Adjust motor current in real-time based on dynamic load conditions.
b. This configuration allows for precise articulation, improved energy efficiency, and enhanced stall detection, which is critical for surgical use where actuation integrity must be guaranteed. Additionally, feedback data may be logged for post-operative diagnostics or real-time system verification.
c. Communication between the MCU and motor drivers may use serial protocols (e.g., UART or SPI) to facilitate:
   i. Real-time driver configuration (e.g., microstepping resolution, current limits),
   ii. Stall detection via built-in telemetry features (e.g., stallGuard™ or coolStep™),
   iii. Status polling for diagnostics.

In the preferred embodiment, the gearbox assembly—integrated into the proximal portion of the distal module—locks into the main body of the instrument via a bayonet-style twist-lock or quarter-turn latching mechanism, enabling secure, repeatable engagement. The locking system ensures axial fixation while maintaining rotational alignment critical to motor-to-gearbox transmission. A tactile or audible detent mechanism may be provided to confirm full engagement, thereby mitigating the risk of partial insertion or mid-procedural disconnection.

Upon mechanical engagement, the distal gearbox interfaces with:
a. Drive System: The motors housed within the main body are aligned with corresponding input pulleys or drive shafts within the gearbox. Torque is transmitted from the motor output shafts to these pulleys through keyed shafts, splined couplings, or magnetically coupled connectors, depending on the embodiment. The pulleys are rotationally supported within the gearbox and connected to cable-driven actuation systems, which traverse the shaft and terminate at the articulated distal tip. Rotation of each pulley results in axial movement of a corresponding control cable, thereby enabling precise, motor-driven articulation of the grasper jaws or other end-effectors (e.g., pitch, yaw, roll).
b. Electrical Interface: A multipin contact array, preferably composed of gold-plated spring-loaded pogo pins in the body and mating pads or sockets in the gearbox, provides a low-resistance electrical connection. This allows for delivery of electrosurgical energy to distal electrodes or sensors, especially in Electrosurgery Mode, under control of the Microcontroller Unit (MCU).
c. Fluidic Coupling: The instrument incorporates a fluid transport pathway originating from the proximal body, routed through a valved connection interface at the gearbox. Upon engagement, a self-sealing conduit mates with the gearbox's internal fluid lumen, which in turn continues along the shaft to deliver irrigant or suction to the operative site. The connection is sealed with medical-grade O-rings or compression gaskets, ensuring sterility and preventing leakage. An embodiment of this invention may incorporate a tubing coupling with a mechanism that allows for independent rotation of the fluid tubing within the shaft at the base and/or distal end, this feature reduces the possibility of kinks due to twisting of the tube. Possible ways to accomplish this include specialized tubing fittings such as a swivel or quick connect fitting.

The gearbox may optionally include idler pulleys, cable guides, or tensioning elements to maintain optimal cable alignment, tension, and return force characteristics. In certain embodiments, sensors within the gearbox may monitor pulley rotation or cable displacement for closed-loop feedback, allowing dynamic adjustment by the control system.

This architecture enables a motor-body, passive-gearbox configuration in which the motors remain permanently enclosed within the reusable handle assembly, while the gearbox and shaft assembly can be detached, sterilized, or replaced independently. This modularity enhances device longevity, reduces maintenance burden, and facilitates rapid intraoperative tool exchange while preserving high mechanical performance, energy integrity, and sterile barriers.

The laparoscopic instrument includes a distal gearbox assembly configured to interface mechanically and functionally with the main body of the device. The gearbox serves as a multi-functional mechanical and routing hub for electrosurgical energy transfer, fluid transport, and mechanical actuation. Upon engagement with the main housing via a locking mechanism (as described in prior sections), the gearbox establishes electrical, fluidic, and mechanical continuity between the internally housed systems and the distal articulation structures.

The gearbox comprises the following primary subcomponents:

a. Electrosurgical Electrical Connector
a. The gearbox includes an electrical contact interface configured to receive high-voltage current from either an internal isolation transformer or an external electrosurgical generator, as previously described. This connector comprises a high-voltage pin or socket assembly, shielded and electrically isolated from surrounding mechanical components. Upon mating with the main body, spring-loaded or gold-plated contact pads align with a corresponding plug or pad set to establish a secure electrical path to the active electrode or cauterization element located at the distal tip.
b. In one embodiment, this connector includes optical or physical isolation barriers, ensuring patient and operator safety in accordance with IEC 60601-1 standards. The electrical pathway is routed within the gearbox through internal conductive traces or wires, encapsulated in an insulating channel to prevent arc discharge or current leakage.
   b. Fluid Transport Connector
a. The gearbox further includes a fluidic coupling interface, configured to connect a fluid delivery or suction line originating from the main housing to the internal fluid channel leading toward the distal end. The interface may be implemented using a self-sealing male or female barb connector, luer-lock fitting, or a quick-connect valve assembly, each designed to maintain leak-proof coupling during engagement and disengagement.
b. The connector may be surrounded by a gasket or O-ring seal, and in some embodiments includes a check valve or anti-backflow element within the gearbox side to prevent contamination during disconnection. Fluid is routed within the gearbox via rigid or flexible medical-grade tubing, and may be bifurcated to serve both suction and irrigation channels if required by the instrument mode.
   c. Pulley and Cable Assembly
a. The gearbox includes a series of rotatable pulleys, each supported by low-friction bearings or bushings, and configured to receive mechanical torque from the motors located within the main body. Each pulley is connected to a corresponding tensioned cable, which traverses through cable guides and sheathings along the length of the distal shaft to the articulated end-effector.
b. Each pulley is matched to a specific degree of motion (e.g., pitch, yaw, grasper open/close, roll), and may incorporate cable take-up mechanisms, tensioning springs, or routing features to maintain constant pre-load and backlash-free movement of the cables during dynamic motion. The pulley diameters, groove profiles, and axis orientation are optimized to provide precise actuation ratios and repeatable displacement of the distal components.
   d. Motor-to-Pulley Coupling Interface
a. The gearbox is further equipped with a motor input interface designed to mechanically couple with the output shafts of the motors housed in the main body. Each input port includes a female coupling receptacle configured to receive a tapered shaft, spline shaft, D-shaft, or magnetic clutch driven by the corresponding motor. During device assembly or operation, insertion of the gearbox into the main housing results in automatic axial and rotational alignment of each motor output with its corresponding pulley shaft.
b. In some embodiments, floating couplers, compliant sleeves, or magnetic detent couplings are employed to tolerate slight misalignments and reduce wear on engagement. Torque is transferred from the motor shaft to the gearbox pulley through direct contact or an intermediary gear/pulley mechanism enabling precise control of distal articulation via motor-driven cable displacement.

Collectively, these components form a modular, detachable gearbox subsystem that enables rapid sterilization, efficient mechanical actuation, and seamless connectivity with the laparoscopic instrument's internal architecture. The design ensures repeatable alignment, leak-proof fluid connections, safe electrical isolation, and robust mechanical performance during high-precision minimally invasive procedures.

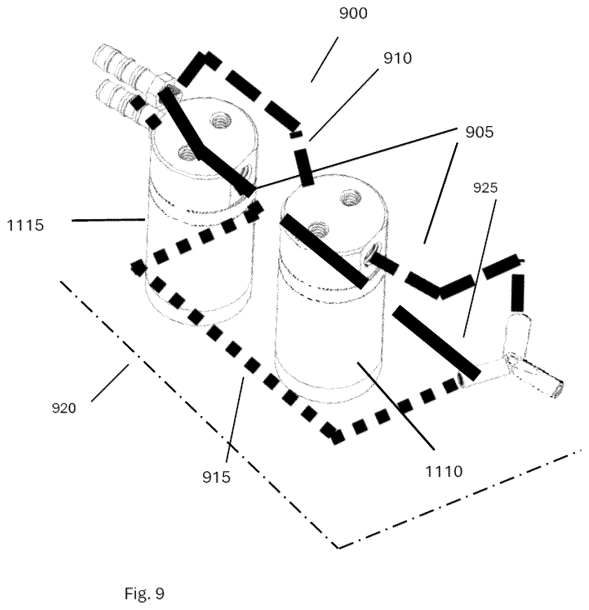

The laparoscopic instrument 200 incorporates a fluid transport system 900 to enable irrigation, suction, and other fluid-related functions during surgical procedures. FIG. 9 illustrates the fluid transport system 900, revealing its components and pathways.

In some cases, the fluid transport system 900 may include multiple flow paths for directing different media through the laparoscopic instrument 200. A fluid flow tubing 905 may provide a main channel through which fluids can be transported. The fluid transport system 900 may incorporate a water flow path 910 to enable the delivery of irrigation fluid, while a suction flow path 915 may allow for the removal of fluids and debris from the surgical site.

The fluid transport system 900 may include an electrical path 920 for transmitting cauterization electricity and a power distribution path 925 for delivering electrical power to various components within the laparoscopic instrument 200. Solenoid 1110 and suction output solenoid 1115 direct the flow of fluids within the system, connecting to a fluid flow tubing 905 that extends through the laparoscopic instrument 200.

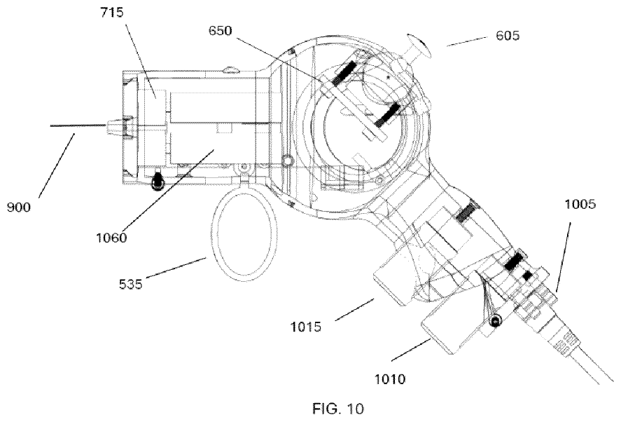

FIG. 10 illustrates a section view of the laparoscopic instrument 200 assembly, showing the internal configuration and routing of various components. The fluid flow tubing 905 may extend through the laparoscopic instrument 200. The laparoscopic instrument 200 may incorporate barbed fittings 1005 to enable secure fluid connections.

In some cases, the laparoscopic instrument 200 may include a motherboard PCB 650 to provide electronic control functionality for the fluid transport system 900. The motherboard PCB 650 may interface with sensors and actuators to regulate fluid flow based on user inputs and operational modes.

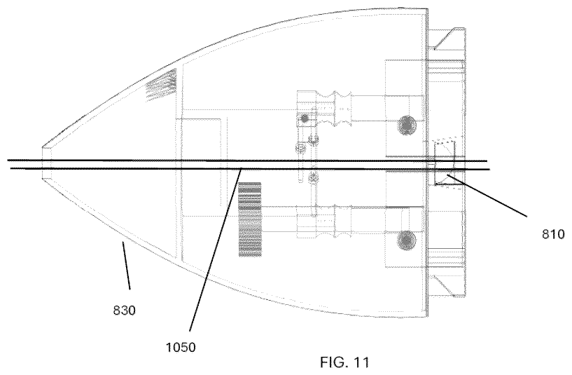

FIG. 11 depicts a section view of a portion of the laparoscopic instrument 200, showing internal components. The laparoscopic instrument 200 may incorporate control cables 1050 extending through the section, which may facilitate mechanical actuation and control of the distal end components. The fluid interface 810 and control cables 1050 may be arranged in a configuration that allows for both fluid transport and mechanical control functions to operate independently within the confined space of the laparoscopic instrument 200 shaft.

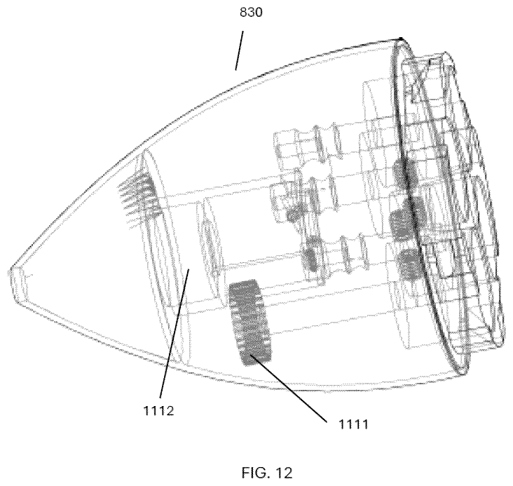

The fluid transport system 900 may utilize solenoid valves to control fluid flow. FIG. 12 illustrates a section view of the second gearbox housing 830 showing internal components, including a water intake solenoid 1110 and a suction output solenoid 1115. The water intake solenoid 1110 and suction output solenoid 1115 may be positioned within the second gearbox housing 830 to control fluid flow through the laparoscopic instrument 200.

In some cases, the water intake solenoid 1110 may regulate the flow of irrigation fluid into the system, while the suction output solenoid 1115 may control the evacuation of fluid through the suction pathway. The second gearbox housing 830 may provide structural support and containment for these fluid control components.

The fluid transport system 900 may enable coordinated control of irrigation, suction, and electrical energy delivery through the laparoscopic instrument 200. The fluid flow tubing 905, water flow path 910, and suction flow path 915 may be arranged to maintain separation between different media while allowing selective activation based on the operating mode selected by the user.

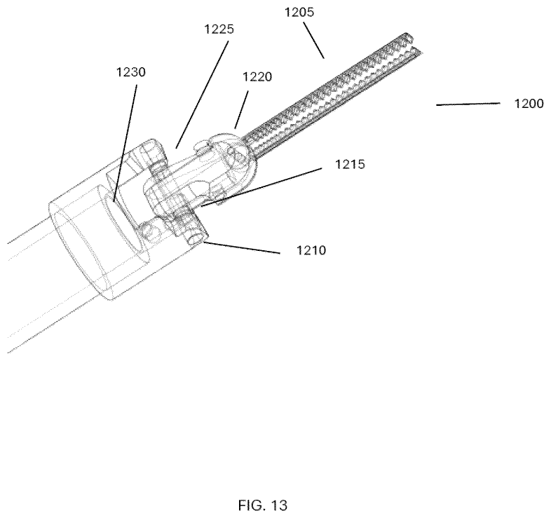

The laparoscopic instrument 200 may incorporate a distal assembly 1200 at its end to enable precise manipulation and control during surgical procedures. FIG. 13 is a view of the distal assembly 1200, revealing its key components and structure.

In some cases, the distal assembly 1200 may include a bipolar grasper 1205 positioned at its distal end. The bipolar grasper 1205 may be designed to grasp, manipulate, and potentially apply electrosurgical energy to tissue during surgical procedures. A wrist control mechanism 1210 may be positioned proximally to the bipolar grasper 1205, enabling articulation of the distal portion of the laparoscopic instrument 200.

The distal assembly 1200 may incorporate a fluid port 1215 for delivering or removing fluids during surgical procedures. This fluid port 1215 may connect to the fluid flow tubing 905 within the laparoscopic instrument 200, allowing for irrigation or suction functions depending on the selected operational mode.

In some cases, the distal assembly 1200 may include a grasper actuator 1220 that controls the opening and closing movements of the bipolar grasper 1205. The grasper actuator 1220 may be mechanically linked to the control cables 1050 that extend through the laparoscopic instrument 200, translating user inputs from the trigger 535 into precise grasper movements.

A grasper rotation mechanism 1225 may be incorporated into the distal assembly 1200, allowing rotational movement of the distal components. The grasper rotation mechanism 1225 may enable the surgeon to orient the bipolar grasper 1205 as needed during a procedure without rotating the entire laparoscopic instrument 200.

The distal assembly 1200 may also feature a retraction tube 1230. In some cases, the retraction tube 1230 may enable the bipolar grasper 1205 to retract when performing suction or irrigation functions through the fluid port 1215. This retraction capability may help maintain a clear fluid pathway and prevent interference between the bipolar grasper 1205 and fluid flow during these operations.

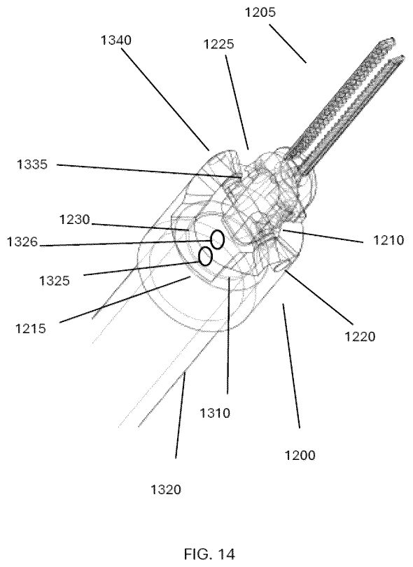

FIG. 14 provides another section view of the distal assembly 1200, revealing additional components and structural details. The distal assembly 1200 may incorporate a base ring 1310 that provides structural support and serves as a mounting point for various components.

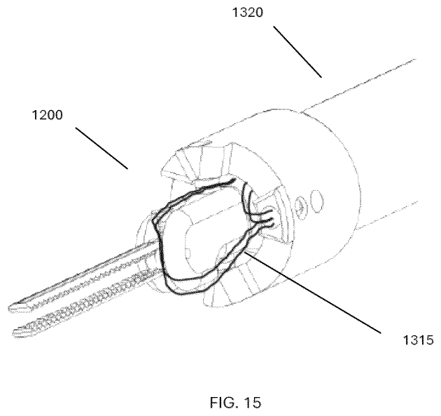

In some cases, the distal assembly 1200 may include control cables 1315 (FIG. 15) that extend through the laparoscopic instrument 200 for actuating different movements. These control cables 1315 (FIG. 15) may connect to the various articulation and actuation mechanisms within the distal assembly 1200, translating motor movements from the second gearbox housing 830 (FIG. 8) into precise control of the bipolar grasper 1205 and other distal components.

A distal shaft 1320 may house internal components and provide structural support for the distal assembly 1200. The distal shaft 1320 may connect the distal assembly 1200 to the main body of the laparoscopic instrument 200, housing the control cables 1315 and fluid pathways.

The distal assembly 1200 may include dedicated channels for fluid management. Specifically, a suction channel 1325 and an irrigation channel 1326 may enable fluid delivery and removal during surgical procedures. These channels may connect to the fluid flow tubing 905 and water flow path 910 within the laparoscopic instrument 200, allowing for controlled fluid management at the surgical site.

An articulation joint 1335 may be incorporated into the distal assembly 1200, allowing for controlled movement of the distal end. This articulation joint 1335 may enable multi-axis movement of the bipolar grasper 1205, enhancing the surgeon's ability to navigate and operate within confined surgical spaces.

In some cases, the distal assembly 1200 may include a pivot shaft 1340 that facilitates articulated motion of the bipolar grasper 1205. The pivot shaft 1340 may serve as a fulcrum point for the articulation movements, working in conjunction with the control cables 1315 to enable precise positioning of the bipolar grasper 1205.

FIG. 15 provides a detailed view of the control cables 1315 within the distal assembly 1200. The control cables 1315 may be routed through internal channels or passages within the distal shaft 1320. These control cables 1315 may be thin, flexible cables that enable mechanical transmission of movement from the second gearbox housing 830 to the various components of the distal assembly 1200.

The arrangement of components within the distal assembly 1200 may allow for independent operation of mechanical actuation, fluid transport, and energy delivery functions. This integration of multiple functionalities into the distal assembly 1200 may contribute to the versatility and precision of the laparoscopic instrument 200 during minimally invasive procedures.

The laparoscopic instrument includes a distal end assembly configured to perform fine mechanical manipulation, fluid management, and energy delivery functions during minimally invasive surgical procedures. The distal end is operatively coupled to the gear box body via an elongated shaft that houses mechanical transmission elements, fluid conduits, and electrical leads. The distal end comprises three primary functional subsystems:

a. Articulated End-Effector

In one embodiment, the distal tip of the instrument comprises an articulated end-effector, such as a grasper, dissector, or forceps. The end-effector is actuated via a cable-driven mechanism, wherein one or more control cables extend from pulleys located in the gearbox (as described in Section IV) through cable channels or sheathings along the instrument shaft, terminating at articulation points on the distal elements.

The articulated end may provide multiple degrees of freedom, including:
a. Grasping motion (open/close of jaws),
b. Pitch and yaw articulation (bending in one or more orthogonal planes),
c. Axial roll (rotation about the longitudinal axis of the shaft).

In some embodiments, the articulation joints include low-friction pivot points or ball-and-socket mechanisms, tensioned to eliminate slack while maintaining smooth, responsive movement. Cable displacement caused by motor-driven pulley actuation results in precise mechanical articulation of the distal structure in real-time.

b. Fluid Transport Pathway

The distal end includes an internal fluid transport conduit in communication with the fluid coupling in the gearbox. In one embodiment, the conduit comprises a flexible or semi-rigid medical-grade tube, routed along the interior wall of the shaft, terminating at one or more exit ports on the distal end-effector.

The exit ports may be located:
a. Proximate to the grasping tip,
b. Along the shaft near the joint,
c. Or distributed in a spray orifice pattern.

Depending on the selected mode of operation (Suction or Irrigation), the trigger input opens the corresponding solenoid valve (as described in Section IV), enabling bidirectional fluid flow—either delivering sterile irrigation fluid or drawing biological material through the suction path.

The fluid pathway may include one-way valves, flow restrictors, or inline pressure sensors to maintain sterility, prevent reflux, and ensure controlled delivery

c. Electrosurgical Cauterization Mechanism

In another embodiment, the distal end includes an integrated cauterization mechanism, designed to deliver electrosurgical current to tissue under user control. The mechanism comprises an active electrode terminal positioned at or near the tip of the grasper or blade, connected via a high-voltage lead routed from the electrosurgical power system (internal or external, as previously described).

Electrosurgical current is delivered in Electrosurgery Mode, activated via a user interface such as a joystick press or designated button. The current is modulated and gated by the control system, and only flows when:
a. The device is locked into Electrosurgery Mode,
b. The operator initiates activation, and
c. Safety interlocks are satisfied (e.g., proper engagement, voltage presence).

The electrode may be constructed from platinum-iridium, stainless steel, or other conductive biocompatible materials, and may be integrated into:
a. A jaw surface (e.g., for monopolar cauterization),
b. An isolated contact point (e.g., bipolar configuration),
c. Or a replaceable tip insert.

The design ensures thermal isolation of non-active components and may include insulation barriers or ceramic supports to prevent unintended energy dispersion.

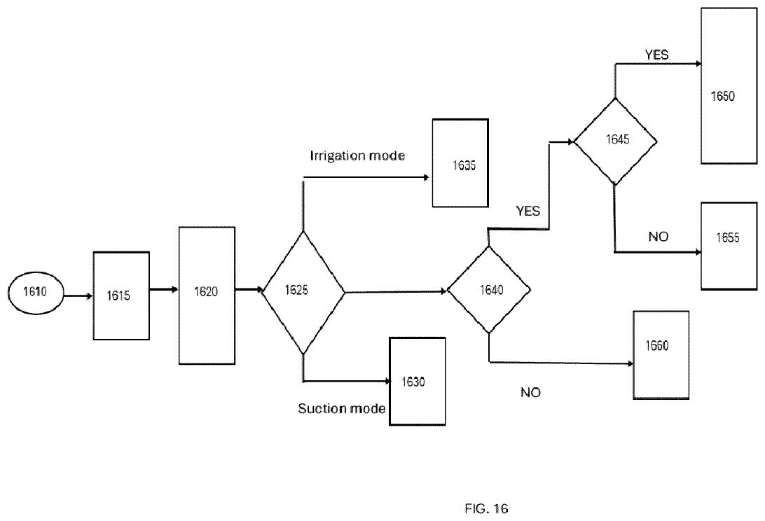

The laparoscopic instrument 200 may incorporate various control processes and operations to enable its multiple functions. These processes may include trigger actuation, electrocautery control, and motor-pulley coupling. FIG. 16 illustrates a flowchart showing a process for operating the laparoscopic instrument 200.

The process may begin at a step 1610 where the user starts the laparoscopic instrument 200. At a step 1615, the user may input longitudinal displacement of the trigger 535. The process may continue to a step 1620 where the hall effect sensor 520 senses magnet displacement within the trigger 535. At a step 1625, the hall effect sensor 520 may interpret the longitudinal displacement and send an actuation signal to a Micro Control Unit.

In all embodiments, the laparoscopic instrument comprises a bidirectional analog trigger mechanism configured to detect user-applied displacement in both the inward (compressive), outward (releasing) directions, and lateral direction. The same physical trigger is utilized across all operational modes, with its functional output dynamically reassigned based on the selected surgical mode. This reassignment is managed by an onboard Microcontroller Unit (MCU) in response to input from the mode selection interface.

The trigger mechanism is equipped with one or more Hall effect sensors operatively positioned to detect real-time changes in magnetic flux corresponding to the degree of displacement. These sensors produce an analog electrical signal that is interpreted by the MCU to generate context-specific outputs, based on the mode currently active.

In some cases, the process may branch based on the operating mode of the laparoscopic instrument 200. If the laparoscopic instrument 200 is in suction mode, at a step 1630 the Micro Control Unit may increase or decrease suction. If the laparoscopic instrument 200 is in irrigation mode, at a step 1635 the Micro Control Unit may increase or stop irrigation flow.

The process may then move to a step 1640 where the Micro Control Unit determines if the bipolar grasper 1205 is at limits of motion. This may lead to a step 1645 where control transfers to determine if the bipolar grasper 1205 is closed at a step 1650.

In some cases, if the bipolar grasper 1205 is closed, the process may move to a step 1655 where the Micro Control Unit stalls the motors at maximum safe grasper pressure until release. If at the step 1640 the bipolar grasper 1205 is not closed, the process may proceed to a step 1660 where the Micro Control Unit implements grasper activation.

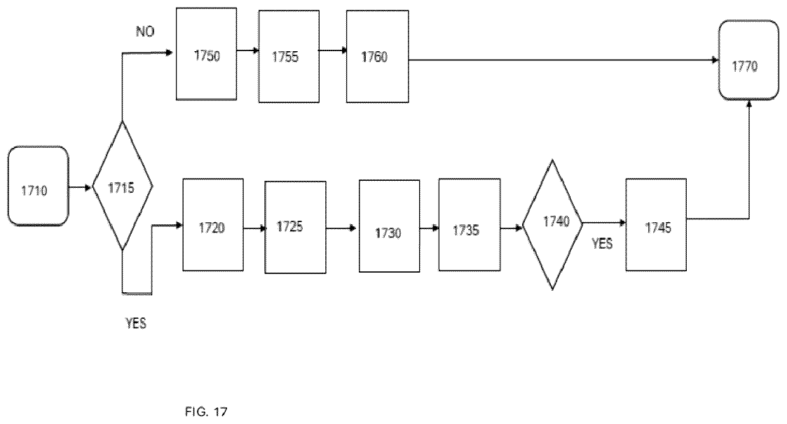

FIG. 17 illustrates a flowchart of an electrocautery control process. The process may begin at a step 1710, where the electrocautery sub-process is initiated. At a step 1715, the laparoscopic instrument 200 may determine if it is in electrocautery mode.

In some cases, if the laparoscopic instrument 200 is in electrocautery mode, the process may move to a step 1720 where a user press listener is activated in the software. The process may then proceed to a step 1725, where the user moves the joystick 605 with magnetic core. At a step 1730, the hall effect sensor 520 may determine displacement and output a signal. The process may continue to a step 1735 where appropriate pitch and yaw rotation of the distal assembly 1200 is determined. At a step 1740, the laparoscopic instrument 200 may check if the user has activated the electrosurgery button 640. Upon button activation, the process may move to a step 1745 where the electrocautery system is activated.

If at the step 1715 the laparoscopic instrument 200 is not in electrocautery mode, the process may follow a different path. It may move to a step 1750 where the user moves the joystick 605 with magnetic core. The process may then proceed to a step 1755, where the hall effect sensor 520 determines displacement and outputs a signal. At a step 1760, the appropriate pitch and yaw rotation of the distal assembly 1200 may be determined.

Both paths may converge at a step 1770, where the sub-process ends. The flowchart shows how the laparoscopic instrument 200 may process joystick inputs differently based on whether electrocautery mode is active or inactive.

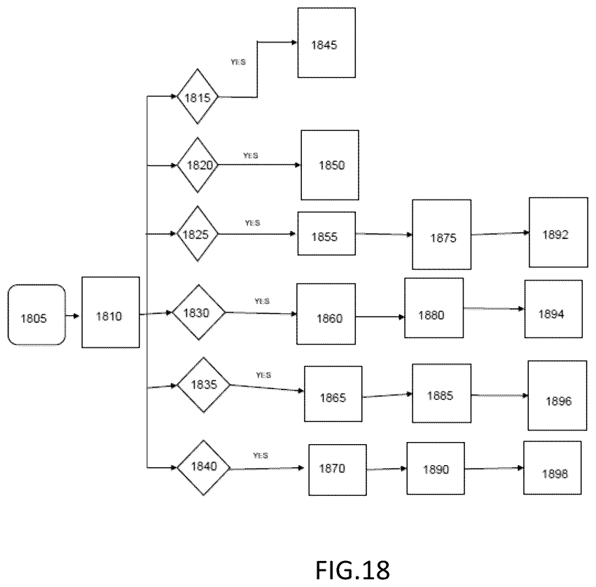

FIG. 18 illustrates a flowchart showing a motor-pulley coupling interface operation process. The process may begin at a step 1805 and proceed to a step 1810 where the laparoscopic instrument 200 determines if the gearbox housing. The laparoscopic instrument 200 may incorporate various control processes and operations to enable its multiple functions. These processes may include trigger actuation, electrocautery control, and motor-pulley coupling. FIG. 16 illustrates a flowchart showing a process for operating the laparoscopic instrument 200.

The process may begin at a step 1610 where the user starts the laparoscopic instrument 200. At a step 1615, the user may input longitudinal displacement of the trigger 535. The process may continue to a step 1620 where the hall effect sensor 520 may sense magnet displacement within the trigger 535. At a step 1625, the hall effect sensor 520 may interpret the longitudinal displacement and send an actuation signal to a Micro Control Unit (MCU).

The process may then branch based on the operating mode. If in suction mode, at a step 1630 the MCU may increase or decrease suction. If in irrigation mode, at a step 1635 the MCU may increase or stop irrigation flow.

The process may then move to a step 1640 where the MCU may determine if the bipolar grasper 1205 is at limits of motion. This may lead to a step 1645 where control transfers to determine if the bipolar grasper 1205 is closed at a step 1650.

If the bipolar grasper 1205 is closed, the process may move to step 1655 where the MCU may stall the motors at maximum safe grasper pressure until release. If at step 1640 the bipolar grasper 1205 is not closed, the process may proceed to a step 1660 where the MCU may implement grasper activation.

FIG. 17 illustrates a flowchart of an electrocautery control process. The process may begin at a step 1710, where the electrocautery sub-process is initiated. At a step 1715, the laparoscopic instrument 200 may determine if it is in electrocautery mode.

If the laparoscopic instrument 200 is in electrocautery mode, the process may move to a step 1720 where a user press listener may be activated in the software. The process may then proceed to a step 1725, where the user may move the joystick 605 with magnetic core. At a step 1730, the hall effect sensor 520 may determine displacement and output a signal. The process may continue to a step 1735 where appropriate pitch and yaw rotation of the distal assembly 1200 may be determined. At a step 1740, the laparoscopic instrument 200 may check if the user has activated the button. Upon button activation, the process may move to a step 1745 where the electrocautery system may be activated.

If at step 1715 the laparoscopic instrument 200 is not in electrocautery mode, the process may follow a different path. It may move to a step 1750 where the user may move the joystick 605 with magnetic core. The process may then proceed to a step 1755, where the hall effect sensor 520 may determine displacement and output a signal. At a step 1760, the appropriate pitch and yaw rotation of the distal assembly 1200 may be determined.

Both paths may converge at a step 1770, where the sub-process ends.

FIG. 18 illustrates a flowchart showing a motor-pulley coupling interface operation process. The process may begin at a step 1805 and proceed to a step 1810 where the laparoscopic instrument 200 may determine if the second gearbox housing 830 is in a default state.

From step 1810, the process may branch into multiple parallel paths to check different activation states:
Suction initiation check at a step 1815
Irrigation initiation check at a step 1820
Roll rotation initiation check at a step 1825
Grasper activation check at a step 1830
Yaw rotation initiation check at a step 1835
Pitch rotation initiation check at a step 1840

If suction is initiated at step 1815, the process may move to a step 1845 where the hand controller valve may activate and fluid may route out through the fluid port 1215.

If irrigation is initiated at step 1820, the process may proceed to a step 1850 where the hand controller valve may activate and fluid may route in through the fluid port 1215.

For roll rotation initiation at step 1825, the process may move to a step 1855 to check roll motor actuation, then to a step 1875 where motors may couple to respective pulleys, and finally to a step 1892 where surgical steel cables may route through the distal shaft 1320.

When grasper activation occurs at step 1830, the process may move to a step 1860 to check motor actuation, then to a step 1880 for pulley rotation, and a step 1894 for grasper operation.

For yaw rotation at step 1835, the process may proceed to a step 1865 for motor actuation verification, then to a step 1885 for pulley rotation, and a step 1896 for cable actuation to the distal assembly 1200.

If pitch rotation is initiated at step 1840, the process may move to a step 1870 to verify pitch motor activation, then to a step 1890 for pulley rotation, and finally to a step 1898 where cables may drive action to the distal assembly 1200.

These control processes and operations may enable the various functions of the laparoscopic instrument 200, allowing for coordinated control of mechanical actuation, fluid management, and energy delivery during surgical procedures.

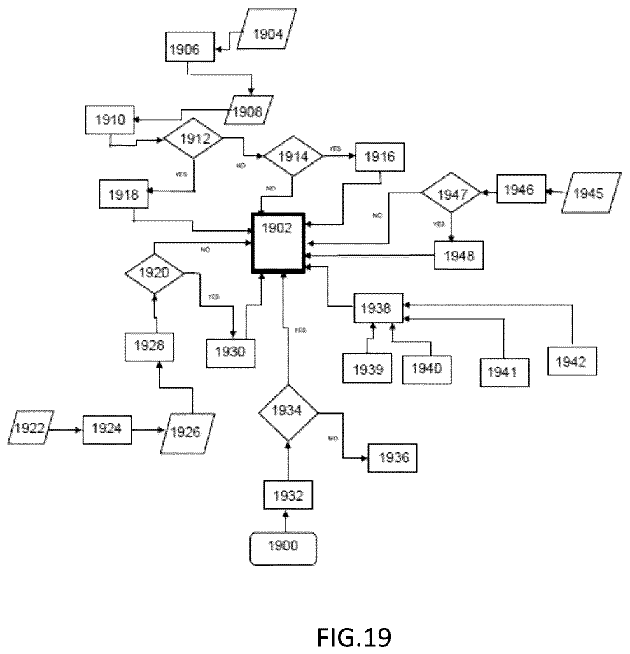

FIG. 19 illustrates a flowchart of a microcontroller unit (MCU) control process start is step 1900 for the laparoscopic instrument 200. The process start 1900 transfers control to step 1932 where the laparoscopic instrument 200 is powered on. The process may then move to a step 1934 where the laparoscopic instrument 200 checks if the second gearbox housing 830 and electrical surgery unit (ESU) connection is detected.

In some cases, if the connection is detected, control may pass to an MCU idle state at a step 1902. If not detected, control may move to a step 1936 where the MCU may send a signal to flash the indicator light 405 indicating invalid use.

The process may include multiple parallel input paths from the MCU idle state. When joystick movement is detected at a step 1922, the hall effect sensor 520 may detect motion at a step 1924, generating sensor data at a step 1926. The MCU may process the joystick input at a step 1928. At a step 1920, the laparoscopic instrument 200 may check if the distal assembly 1200 is within safe motion limits. If within limits, signals may be sent to motor drivers to rotate motors at a step 1930, returning to MCU idle at step 1902.

The process may monitor for mode activation through buttons:
Irrigation mode activation at a step 1939
Suction mode activation at a step 1940
Electrocautery mode activation at a step 1941
Standard mode activation at a step 1942

When any mode is activated, a step 1938 may detect the mode change and update the indicator light 405 before returning to MCU idle at step 1902.

For electrosurgery activation, the process may check for foot pedal or joystick button press at a step 1945. The MCU may detect this input at a step 1946 and verify electrocautery mode at a step 1947. If verified, a step 1948 may send signals to enable ESU power flow before returning to idle.

The process may also monitor trigger motion at a step 1904, where hall effect sensing may occur at a step 1906 and data may be recorded at a step 1908. The MCU may process trigger input at a step 1910 and check for suction/irrigation modes at a step 1912. For mechanical actuation, a step 1914 may verify grasper motion limits before sending motor rotation signals at a step 1916.

In some cases, the MCU control process may enable coordinated operation of the various subsystems within the laparoscopic instrument 200, including fluid management, mechanical actuation, and energy delivery. The process may allow for real-time response to user inputs and dynamic switching between operational modes, enhancing the versatility and precision of the laparoscopic instrument 200 during minimally invasive procedures.

The laparoscopic instrument comprises an embedded Microcontroller Unit (MCU) mounted on a compact custom-printed circuit board (PCB) located within the instrument's central housing. The MCU functions as the primary control logic unit, coordinating all actuation, sensing, user input interpretation, and safety protocols associated with the instrument's operation.
a. MCU Board Configuration
   a. In one embodiment, the MCU is mounted on a single-layer or multi-layer compact PCB, dimensioned to fit within the spatial constraints of the instrument's ergonomic housing. The PCB is mechanically supported by standoffs or integrated slots within the housing and may be fabricated using high-density interconnect (HDI) design rules to minimize board footprint while maximizing routing capacity.
      i. The PCB includes:
         1. A central microcontroller or microprocessor (e.g., ARM Cortex-M series),
         2. Power conditioning components (e.g., LDOs or buck converters),
         3. Logic-level shifters (if required),
         4. Connector headers or flex circuit interfaces for motor drivers, sensors, and user input components.
   b. Peripheral Connections and Interfaces
      i. The MCU is electrically and logically connected to the following subsystems:
         1. Motor Driver Interfaces:
         a. The MCU provides digital control signals to four motor driver circuits, each corresponding to a respective micromotor for grasper movement, pitch, yaw, and roll. In one embodiment, these signals are conveyed via step/direction lines; in another embodiment, they use a serial protocol such as SPI, UART, or I²C for configuration and telemetry.
   c. User Input Components:
      i. Mode Selection Buttons: Four momentary-contact push buttons (e.g., SMD tactile switches) electrically coupled to the MCU via GPIO pins, used to switch between Normal, Suction, Irrigation, and Electrosurgery modes.
      ii. Roll Control Buttons: Two lateral buttons used to initiate clockwise and counterclockwise roll commands, also routed to the MCU through digital input pins.
      iii. Joystick Interface: A two-axis Hall effect joystick, including analog output signals for pitch and yaw control and a digital press-to-click input for electrosurgical energy activation. The analog channels are routed to the MCU's ADC (analog-to-digital converter) inputs, while the press input is routed to a digital interrupt pin. The joystick-based control module is configured for multi-axis manipulation of the distal end-effector, specifically enabling articulated control over pitch and yaw degrees of freedom of the grasper assembly.
      iv. Trigger Mechanism: A Hall effect or displacement-based sensor mounted to the trigger, producing either analog or digital signals based on the degree of depression or lateral displacement. These are routed to the MCU for context-specific interpretation (e.g., grasper control, fluid valve activation).
      v. Safety Sensors and Feedback: The MCU receives input from temperature sensors, current monitors, and optional position encoders (in a closed-loop embodiment) to determine fault conditions and to initiate automatic system shutdown or fail-safe behavior if predefined thresholds are exceeded.
   c. Firmware and Control Logic
      vi. The MCU executes embedded firmware implementing:
         1. Motion control algorithms (e.g., trapezoidal acceleration curves, S-curve smoothing),
         2. Input polling and debouncing for all mechanical buttons and trigger inputs,
         3. Mode switching logic, dynamically remapping trigger and joystick functions based on current operating mode,
         4. Safety watchdog routines, including thermal shutdown, stall detection, and comms fault resets,
         5. State management and telemetry, with optional UART or USB interface for configuration or diagnostics.

In certain embodiments, the MCU firmware may be designed for field programmability, allowing software updates via a wired interface or programming header.
d. Electromagnetic and Sterilization Considerations
   a. The MCU PCB is designed with electromagnetic compatibility (EMC) shielding, with grounded copper pours and filtering capacitors at all external interface ports. The board and associated components are selected for sterilization resilience, supporting low-temperature hydrogen peroxide plasma or ethylene oxide (EtO) protocols where applicable.

As one can envision the hilt and shaft may be designed as separable modules, facilitating reusability of the electronic and mechanical components while allowing the shaft—particularly the sections exposed to biofluids—to be discarded or sterilized independently. In one embodiment, the outer housing of the hilt is configured to split open, providing access to internal tubing and wiring for inspection, cleaning, or replacement.

This modular, multifunctional design aims to reduce operative time, enhance ergonomic control, and improve surgical precision by enabling surgeons to perform grasping, articulating, fluid management, and energy delivery tasks with a single, compact tool.

FIG. 2 illustrates an orthogonal side view of a laparoscopic instrument 200 and the surgical instrument is connected to a computerized control system that displays the hierarchical structure of components and features used within the application to manage the assembly.

FIG. 3 illustrates a browser menu interface for managing a laparoscopic instrument assembly. The browser menu for the laparoscopic instrument s 200 is a user Interface which is a hierarchical structure of components and features used within the application to manage the assembly that include administration as well as operational modules including but not limited to the User interface 300 which has the device name 305, document settings 310, named views 315, origin 320, analysis 325, hand controller 1 330, gearbox 1 335, distal end 1 340, and power supply unit 1 345.

FIG. 4 illustrates an orthogonal view of the laparoscopic instrument of FIG. 2 and it shows laparoscopic instrument 200 body, LED indicators 405, Joystick 415, mode selection buttons 420, electrosurgery connection 425, barbed fittings 430 and rotate buttons 410.

The mode selection subsystem disposed on the exterior surface of the device handle, herein referred to as the mode switch interface. This subsystem is configured to enable the user to selectively engage one of a plurality of operative modes, each corresponding to a distinct surgical function performed by the instrument.

In the preferred embodiment, the mode switch interface consists of four discrete, user-actuated buttons, spatially arranged on the lateral surfaces of the instrument body in proximity to the thumb-operated joystick. The spatial configuration is such that two buttons are located on each lateral side of the joystick, enabling intuitive, low-latency access by the operator's thumb during surgical manipulation. The tactile proximity of these buttons to the joystick facilitates seamless transitions between modes without necessitating repositioning of the user's grip, thereby enhancing ergonomic efficiency and minimizing intraoperative interruption.

Each button corresponds uniquely to one of the following operating modes:

The mode buttons 420 comprise of

Normal Mode (White): wherein actuation of the primary trigger mechanism results in mechanical opening and closing of the distal grasper jaws.

Suction Mode (Red): wherein actuation of the trigger mechanism engages a suction channel integrated within the instrument shaft to evacuate fluids or debris.

Irrigation Mode (Blue): wherein actuation of the trigger mechanism initiates the controlled delivery of an irrigant through an internal channel to the surgical site.

Electrosurgical Mode (Yellow): wherein actuation of the trigger mechanism continues to control grasper actuation, while either depression of the joystick or pressing on a foot pedal trigger as is commonly found in operating rooms, functions as a switch for delivering electrosurgical energy through the active tip, such energy being provided by an external generator and controlled via the instrument's internal circuitry.

Each of the four buttons is color-coded to visually denote the respective operative mode to which it is assigned. This color differentiation may optionally comply with ANSI/AAMI or other surgical standards for intuitive recognition (e.g., blue for irrigation, red for electrosurgery, etc.).

The buttons themselves are each coupled with a low-profile push-button cap designed for gloved-hand actuation. Upon depression, the selected switch transmits an electrical signal to an onboard microcontroller or control logic circuit, which in turn reconfigures the functional mapping of the instrument's trigger and/or joystick accordingly.

FIG. 5 illustrates a section view of a trigger 535 assembly mechanism, actuation mechanism has torsional springs 510, torsional spring lock 530, hall effect sensor 520 and bearing 540.

To facilitate this functionality, the instrument is equipped with at least one, and preferably a pair of Hall effect sensors operatively positioned on lateral sides of the trigger body. These sensors are strategically oriented to measure the relative positional displacement of the trigger with respect to a fixed magnetic reference or field-generating element. As the trigger is depressed or released, the changing magnetic field strength is transduced by the Hall effect sensor(s) into an analog electrical signal proportional to the degree of trigger travel.

The analog output of the Hall effect sensors is communicated in real time to a Microcontroller Unit (MCU) integrated within the device. The MCU processes the displacement data and maps it to a corresponding output function, such as adjusting the angular velocity or degree of opening/closing of the distal grasper mechanism. In certain embodiments, the data may further be filtered or interpolated to enhance resolution and responsiveness, thereby enabling proportional and directionally sensitive control of the surgical end-effector.

The trigger-sensing system provides several advantages over conventional mechanical or switch-based actuation systems.

These advantages include:
Enhanced precision and responsiveness in tissue manipulation;
The ability to perform graded force application or progressive jaw actuation;
Improved ergonomics and tactile feedback, enabling more intuitive surgical interactions.

### Surgeon Workflow: Operating the Trigger

Upon insertion of the instrument into the surgical field, the surgeon selects the desired operative mode using the color-coded mode selection buttons located near the thumb-operated joystick. Once a mode is selected, the MCU maps the trigger behavior accordingly:

### In Normal Mode (White):

a. The surgeon depresses the trigger to gradually close the grasper jaws.
b. The degree of closure is proportional to the depth of trigger compression, enabling precise tissue manipulation, retraction, or dissection.
c. Releasing the trigger causes the jaws to open incrementally, unless a locking or holding feature is engaged.

### In Suction Mode (Red):

a. Depressing the trigger opens an internal solenoid valve linked to a suction channel.
b. During this mode, the grasper is automatically retracted into a distal groove in the instrument's end-effector assembly. This retraction reorients the device such that the most distal anatomical surface becomes the fluid-facing orifice. This configuration ensures unobstructed fluid evacuation and minimizes soft tissue interaction.
c. Fluid or debris is evacuated through the shaft as long as the trigger remains depressed.
d. The trigger acts as a flow control switch and does not influence the grasper.

### In Irrigation Mode (Blue):

a. Trigger depression opens a separate solenoid valve connected to an irrigation channel.
b. Again, the distal grasper mechanism automatically recedes into a recessed orientation within the distal end, exposing only the irrigation port as the foremost structure. This facilitates targeted fluid delivery while shielding the grasper mechanism.
c. An irrigant (e.g., saline) is delivered to the operative site through internal tubing.
d. Flow rate may optionally scale with trigger displacement, if implemented.

### In Electrosurgical Mode (Yellow):

a. The trigger continues to actuate the mechanical grasper (as in Normal Mode).
b. Energy delivery is not linked to the trigger, but is controlled by a separate activation method—either by pressing down on the joystick or using an OR-standard foot pedal.
c. This separation ensures precise coordination between tissue manipulation and energy application.

Throughout use, the surgeon's hand remains in a stable grip, with the index finger controlling the trigger while the thumb accesses mode buttons and the joystick. This configuration allows seamless transitions between functions with minimal disruption to hand positioning or operative flow.

FIG. 6A illustrates a view of a joystick control assembly showing joystick control system 600 having the joystick 605, CW roll buttons 610, irrigation button 625, suction button 630, normal operation button 635, Electrosurgery 640.

FIG. 6B is a view of the joystick control assembly of FIG. 6A showing Joystick Control system 600 having the joystick 605, PCB mounting screws 615, motor power out 620, hall effect sensor 655, switching power supply unit connection (PSU) 645 and Mother Board PCB 650.

The laparoscopic instrument uses a joystick-based control module which is configured for multi-axis manipulation of the distal end-effector, specifically enabling articulated control over pitch and yaw degrees of freedom of the grasper assembly. The joystick is mounted within the housing shell of the instrument, in a location ergonomically accessible to the operator's thumb during standard instrument handling.

To achieve a complete range of articulation at the distal end of the laparoscopic instrument, including axial rotation (roll) of the elongate shaft or distal effector, the laparoscopic instrument comprises a control system for initiating and regulating rotational motion of the distal segment about its longitudinal axis.

In one embodiment, rotation is actuated by one or more user interface inputs—preferably a pair of dedicated roll buttons—disposed on the instrument body in close proximity to the joystick interface. These buttons may be implemented as momentary tactile switches, allowing for intuitive bidirectional roll control when pressed individually or sequentially. Upon actuation, the associated button transmits a signal to the Microcontroller Unit (MCU), which in turn engages a miniature actuator (e.g., a rotational micro-motor, servo, or shape memory alloy) embedded in the shaft housing or proximal actuation assembly, thereby effectuating rotation of the distal segment.

In an alternative embodiment, control over axial rotation may also be effected by integration of lateral (horizontal) displacement sensing within the primary trigger mechanism. This is achieved by augmenting the trigger with additional Hall effect sensors or capacitive sensors positioned orthogonally to the direction of inward/outward trigger displacement. These sensors are calibrated to detect sideways translational movement of the trigger component within its housing slot, such that lateral pressure applied by the user's index finger produces a corresponding signal indicative of intended roll direction and magnitude.

The lateral displacement data is transmitted to the MCU, which maps the horizontal input to a rotational velocity or step command for the distal shaft. In certain embodiments, the system may incorporate a threshold zone or deadband, ensuring that only deliberate lateral movements are interpreted as roll commands, thereby preserving intuitive operation and avoiding unintentional actuation during standard grasping functions.

This dual-mode control scheme-comprising dedicated roll buttons and lateral trigger sensing-provides redundancy and flexibility in surgeon preference, enhancing dexterity, precision, and responsiveness during minimally invasive surgical maneuvers. Moreover, it enables continuous, proportional control over axial shaft rotation without requiring hand repositioning or additional instruments, thereby improving operative efficiency and ergonomic control.

The laparoscopic instrument includes a modular distal-end assembly configured to mechanically and functionally interface with the main instrument body, wherein the body houses one or more electric motors responsible for actuation of the surgical tool's distal components. The interface between the body and the distal assembly is designed to facilitate precise alignment and robust coupling of multiple subsystems, including mechanical drive, electrical energy delivery, and fluidic flow.

In the preferred embodiment, the joystick utilizes contactless Hall effect sensing to detect directional displacement. This implementation substantially mitigates or eliminates the phenomenon of stick drift commonly observed in resistive or potentiometric joystick designs, thereby ensuring long-term accuracy, precision, and mechanical reliability over repeated surgical use cycles and sterilization procedures.

The Hall effect sensors within the joystick assembly detect positional changes in the joystick's actuating shaft in orthogonal axes corresponding to pitch and yaw. These analog signals are processed and transmitted to the Microcontroller Unit (MCU) housed within the instrument. The MCU interprets the input to generate corresponding control signals for the actuation of the distal joint(s), enabling real-time, intuitive manipulation of the surgical end-effector in two degrees of freedom.

Additionally, the joystick features an integrated axial press mechanism, operable by depressing the joystick vertically toward the body of the device. This press-to-click functionality is electronically isolated from the directional sensing components and operates as a discrete digital input. In one embodiment, this input is mapped to control electrosurgical current activation when the instrument is placed in Electrosurgery Mode, as determined by the mode selection subsystem described hereinabove. Actuation of the joystick press initiates or modulates the flow of electrosurgical energy to the distal electrodes, subject to predefined safety and interlock conditions managed by the MCU.

The combination of multi-axis Hall effect displacement sensing with a centralized press-to-activate switch allows the joystick to serve as a multifunctional control interface, consolidating movement and energy delivery functions within a single, thumb-operated input device. This configuration contributes to procedural efficiency, reduced cognitive load, and enhanced ergonomic performance during minimally invasive surgical operations.

FIG. 7 illustrates a section view of a gearbox housing including the first gearbox housing 700 from the instrument side of laparoscopic instrument 200 and body connection mechanism having electrosurgery contacts 710, Magnetic shaft couplers 715, fluid outlet to gearbox 720 and twist lock tabs 725.

FIG. 8 illustrates a section view of a gearbox housing showing internal components including the second gearbox housing 830 distal end from the laparoscopic instrument 200 side having electrosurgery connections 805, polymer water tight fluid interface 810, magnetic shaft couplers 815 that connect to the magnetic shaft couplers 715 shown in FIG. 7 and Twist lock slots 825 that connect to the twist lock tabs 725 shown in FIG. 7.

FIG. 9 illustrates a fluid transport system of a laparoscopic instrument including the fluid transport system 900 comprising of fluid flow tubing 905, water 910, suction 915, cauterization electricity path 920 and power line 925, suction out solenoid 1115, water intake solenoid 1110.

FIG. 10 illustrates a section view of a laparoscopic instrument assembly including the micromotors and drivers 1060, fluid transport system 900, magnetic shaft couplers 715, MCU 650, joystick 605, barbed fittings 1005, suction out solenoid 1115 water intake solenoid 1110 and trigger 535.

FIG. 11 illustrates a section view of a portion of the second gearbox housing 830 of the laparoscopic instrument from the distal end of laparoscopic instrument 200 and showing the polymer water tight fluid interface 810, endo wrist cables 1050.

FIG. 12 illustrates a section view of a second gearbox housing 830 showing internal components including gears in the second gearbox housing 830, roil actuation gears 1111 and drive shaft bearing 1112.

FIG. 13 illustrates view of a distal assembly of a laparoscopic instrument comprising of distal end 1200 having bipolar grasper 1205 but the grasper 1205 can be selected from any reasonable configuration, wrist control 1210 which controls the wrist pitch, retraction tube 1230 that allows the grasper to retract when irrigating or suctioning, fluid port 1215 for suction and irrigation, grasper opening and closing 1220 and grasper rotation 1225.

FIG. 14 illustrates another section view of the distal assembly of FIG. 13 showing Distal End 1200 has bipolar grasper 1205 but the grasper 1205 can take any reasonable configuration, wrist control 1210 which controls the wrist pitch, retraction tube 1230 that allows the grasper to retract when irrigating or suctioning, fluid port 1215 for suction and irrigation, grasper opening and closing 1220, pivot shaft 1340, articulation joint 1335, suction channel 1325, irrigation channel 1326, distal shaft 1320, base ring 1310 and grasper rotation 1225.

FIG. 15 illustrates a section view of a portion of the laparoscopic instrument Distal End 1200 showing activation cables 1315 and distal shaft 1320.

FIG. 16 illustrates a flowchart showing a process for operating a laparoscopic instrument. The steps include Step 1610 where the user starts the instrument step 1615; the user inputs the longitudinal displacement of the trigger; step 1620 the Hall Sensor senses the magnet displacement within the trigger. The hall sensor interprets longitudinal displacement and sends actuation signal to the Micro control unit step 1625. If the hall sensor is in suction mode the MCU increase or decreases suction step 1630. If the hall sensor is in irrigation mode the MCU increases or stops irrigation flow step 1635. The MCU then determines if the grasper is closed to limits of motion of the grasper in step 1640 the MCU then transfers control to step 1645 to determine if the grasper is closed step 1650 if the grasper is closed the MCU stalls the motors to maximum safe grasper pressure until release Step 1655 if the Grasper is not closed in step 1640 the grasper is not closed then the Micro control unit implements Grasper activation step 1660.

FIG. 17 illustrates a flowchart of an electrocautery control process wherein the user starts the electrocautery sub process of the system in step 1710 and the system determines if the system is in electrocautery mode step 1715? If it is the MCU transfers control to step 1720 which is the user press listener button in the software then the user moves the joystick with magnetic core step 1725 the system then determines the whether the hall effect sensor is below direct displacement and output signal step 1730, then the system then determines the appropriate pitch and yaw rotation of the distal end step 1735 and then the system determines if the user has activated the button step 1740. Once the button is activated the system activates the electrocautery system step 1745 and ends the sub process step 1770.

FIG. 18 illustrates a flowchart showing a motor-pulley coupling interface operation process. The motor-pulley coupling interface operates the yaw direction of the distal end, actuates the graspers, operates the roll rotation for the distal end, activates the irrigation system and activates the suction system. The process start step 1805 and it then determines if the gear box in the default state step 1810, if the gear box is in the default state the system determines the state of suction-initiated step 1815, the irrigation-initiated step 1820, the roll rotation for distal end initiated step 1825, is the grasper activated step 1830, is the yaw rotation activated step 1835 or is the pitch rotation for the distal end is initiated step 1840.

If the suction-initiated step 1815 is yes then the system activates the hand controller valve and fluid is routed out through the distal port step 1845.

If the irrigation-initiated step 1820 is yes then system activates the hand controller valve and fluid is routed int through the distal port step 1850.

If the roll rotation for distal end is initiated step 1825 is yes then system determines if the roll motor within the hand controller is actuated step 1855. The motors are coupled to respective pulley within the gear box such that the pulley rotates within the gear box step 1875 and then the thin surgical steel cables attached to the pulleys and routed through the device shaft drive action to distal end step 1892.

If the grasper actuated step 1830 then system determines if the two motors are actuated within the hand controller (these are the same two motors that control yaw motion) actuated step 1860. The motors are coupled to respective pulleys within the gear box; the pulleys rotate within the gear box step 1880 and the graspers close or open step 1884.

If the Yaw rotation for distal end is initiated step 1835 then system determines if the two motors are actuated within the hand controller (these are the same two motors that control grasper motion) actuated step 1865. The motors are coupled to respective pulleys within the gearbox; thus, the pulleys rotate within the gear box step 1885 and then the thin surgical steel cables attached to the pulleys and routed through the device shaft drive action to distal end step 1896.

If the pitch rotation for the distal end is initiated step 1840 and the pitch motor within the hand controller is activated step 1870 then the motor to the respective pulleys within the gear box, such that the pulley rotates within the gear box step 1890 and then the thin surgical steel cables attached to the pulleys and routed through the device shaft drive action to distal end step 1898.

FIG. 19 illustrates a flowchart of a microcontroller unit (MCU) control process. The process starts at step 1900 and the device is powered on step 1932. Then the system checks if the gear box and electrical surgery unit (ESU) connection detected step 1934, if yes the control is passed to the MCU idle step 1905 if not the control is passed to step 1936 and the MCU sends signal to flash LED light to indicate invalid use was initiated.

If the user moves the joy stick step 1922 the hall effect sensor near the MCU and below the joystick with ferrite core detects the motion step 1924 which is the HE sensor data step 1926 and the MCU processes input from joystick step 1928 and the system determines if the distal end grasper within safe limits of motion step 1920 if yes the system sends signals to respective motor drivers(s) to rotate motor(s) step 1930 and pass control to step 1902 MCU idle. If not then the system passes control to step 1902 MCU idle.

If the surgeon activates irrigation mode by pressing the button step 1939 or the surgeon activates suction mode by pressing button step 1940 or the surgeon activates electrocautery mode by pressing button step 1941 or the surgeon activates standard mode by pressing button step 1942 the MCU detects mode change and switches configuration/changes LED color to reflect mode step 1938 and then passes control to MCU idle mode step 1902.

If the user presses on foot pedal or presses joystick button step 1945 then the MCU detects the input step 1946 and the system determines if the device is in electrocautery mode step 1947 if yes the system sends a signal to the electrical and optical isolation elements to open flow from the ESU (modern ESU regulates power safely) step 1948 and then passes control to MCU idle mode step 1902. If the device is not on electrocautery mode then passes control to MCU idle mode step 1902.

If the system senses trigger motion from user input (Inward, Outward or Lateral) step 1904 the hall effect sensor detects motion of the ferrite material within the trigger step 1906 and the hall effect sensor data is recorded step 1908. Then the MCU processes input from the trigger step 1910 and the MCU checks that is the system in either suction mode or irrigation mode step 1912. If the yes then the system sends a signal to the respective solenoid valves to adjust flow in the suction or irrigation lines and then passes control to MCU idle mode step 1902. If the result of step 1912 is no then the system determines if the graspers within safe limits of motion for the instrument step 1914 and if yes the system sends a signal to respective motors to rotate step 1916 then passes control to MCU idle mode step 1902. If the result of step 1912 is no then the system passes control to MCU idle mode step 1902.

### Safety and Signal Management

The MCU enforces single-mode operation at all times to prevent cross-interference between subsystems. Trigger signals are debounced, filtered, and checked against system state flags to avoid unintended actuation. Fail-safes are built in to disable fluid or energy delivery in the event of sensor faults or unexpected input patterns.

In preferred embodiments, a power loss results in the trigger returning to a neutral mechanical state, with all solenoid valves defaulting to a closed position and the grasper reverting to a safe-open or locked configuration, depending on system settings.

The laparoscopic instrument includes a low-profile, thumb-operated analog joystick mounted on the dorsal surface of the handle. It is ergonomically positioned to allow continuous access during natural grip without requiring hand repositioning. The joystick provides multidirectional input and is configured to detect:
a. Pitch (Y-axis) movement of the distal end,
b. Yaw (X-axis) movement of the distal end,
c. Z-axis (axial depression) for discrete activation events such as electrosurgical energy delivery.

Internally, the joystick uses a magnet-based contactless displacement sensing architecture (e.g., Hall effect sensors), enabling real-time analog input without reliance on mechanical resistance or potentiometric failure-prone components. The resulting input signals are processed by the system's Microcontroller Unit (MCU), which maps joystick activity to specific motor control outputs or device actions based on the currently selected surgical mode.

### Surgeon Workflow: Intraoperative Joystick Use

### Grip and Baseline Configuration

a. The surgeon's thumb rests naturally on the joystick, enabling access to all directional inputs and the press-to-activate switch. The index finger operates the main trigger, allowing simultaneous mechanical and electronic actuation.

### Distal Tip Articulation (Normal/Electrosurgical Mode)

a. Lateral deflection (X) rotates the distal segment (yaw),
b. Forward/backward deflection (Y) flexes it vertically (pitch),
c. Motion is proportional, continuous, and reversible in real time,
d. Articulation persists during joystick engagement and ceases on release unless otherwise programmed.

### Energy Activation (Electrosurgical Mode)

a. The joystick is pressed axially (Z-axis) to deliver monopolar or bipolar electrosurgical current.
b. Axial depression is interpreted as a digital signal, gated through safety logic to prevent unintended energy discharge.

Activation is visually or haptically confirmed when required by surgical safety protocols.

### Contextual Remapping

a. In modes like Suction and Irrigation, joystick input may be deactivated or reassigned by the MCU to prevent interference or unintended motion.
b. This remapping is entirely software-based and invisible to the user, preserving intuitive operation.

In summary the Laparoscopic instrumentation engineering elements

### 1. Sensor and Input Architecture

a. Hall effect sensors for multi-axis joystick displacement detection (pitch and yaw)
b. Joystick “press-to-click” functionality mapped to electrosurgery activation
c. Analog trigger with Hall effect sensors capable of detecting variable compression depth
d. Lateral displacement sensing via the trigger to control axial shaft rotation (roll)
e. Real-time mapping of sensor outputs to control logic via a microcontroller
f. These features go significantly beyond the general reference to a “joystick” in the provisional and introduce new sensing modalities and control pathways.

### 2. Embedded Control System

a. A centralized microcontroller unit (MCU) coordinating input interpretation, motor actuation, mode switching, and safety interlocks
b. Embedded firmware responsible for executing motion profiles, analog signal interpretation, stall detection, fault protocols, and user input remapping
c. Digital communication protocols including UART and SPI for real-time interface between the MCU, motor drivers, and sensor subsystems
d. Optional field-programmable firmware capability for in-field updates or refinements
e. No control system or software logic architecture was described in the provisional.

### 3. Motor and Gearbox Subsystem

a. Four independent stepper motors controlling pitch, yaw, roll, and grasping movements
b. Closed-loop motor control enabled by encoder feedback (magnetic or optical)
c. Gearbox system containing:
d. Pulley and cable assemblies for motion transmission
e. Tensioners and idler pulleys for maintaining actuation fidelity
f. Motor-to-gear interfaces using splined shafts, floating couplers, or magnetic couplings
g. Mechanical detents and keyed alignment features to ensure secure insertion and engagement between the hilt and disposable shaft
h. The provisional mentioned an “articulated tip” and interchangeable shafts, but did not specify any internal drive mechanism, cable architecture, gearbox configuration, or motor control systems.

### 4. Fluidic System Enhancements

a. Electronic actuation of irrigation and suction using solenoid valves
b. Check valves to prevent retrograde flow within the fluid lines
c. Proportional fluid control linked to trigger displacement depth
d. Modular quick-connect fluid fittings designed for ease of assembly and disassembly
e. Independent rotation of fluid tubes via swivel or anti-kink fittings
f. Fluidic routing embedded within the shaft and gearbox
g. While suction and irrigation were referenced in the provisional, there was no mention of the control architecture, valve design, routing mechanisms, or integration into the modular shaft structure.

### 5. Power System and Electrosurgery Integration

a. Switching power supply converting AC mains (110/220V) to regulated DC output rails (e.g., 12V, 5V)
b. Dedicated power branches for motors, sensors, and digital control subsystems
c. Integration with an external electrosurgical generator via opto-isolated high-voltage input
d. Electrosurgical energy activation gated through joystick press or foot pedal with embedded safety interlocks
e. Internal logic to ensure that electrosurgical current is only delivered under specific conditions (e.g., mode selection, system readiness)
f. The provisional mentions cauterization generally and references LED indicators for modes, but does not describe electrosurgical energy delivery, current routing, interlocks, or safety protocols.

### 6. User Interface and Mode Control

a. Four mode-selection buttons positioned laterally around the joystick for thumb-access control
b. Mode mapping includes Normal (white), Suction (red), Irrigation (blue), and Electrosurgery (yellow)
c. ANSI/AAMI-compliant color coding for visual mode confirmation
d. Tactile button design suitable for use with gloved hands
e. Integration of mode-switching logic into MCU and dynamic remapping of trigger/joystick behavior depending on the selected mode
f. The provisional only refers to a visual LED indicator for the selected operational mode and does not specify user input structure, button placement, color-coding, or electronic remapping logic.

### 7. Safety, Diagnostics, and Maintenance Architecture

a. Thermal sensors, current sensors, and fault detection routines monitored by the MCU
b. Automatic shutdown protocols upon detection of fault conditions (e.g., motor stall, temperature threshold, abnormal current)
c. Optional telemetry for status reporting and post-operative diagnostics
d. Modular split-shell housing allowing for internal inspection and service of tubing and electronics
e. Sterilizable design using high-performance materials such as PEEK and PPSU
f. Snap-fit or luer-lock connectors for replacing internal fluid lines

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, DVD or additional RAM and solid state drives. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

The device disclosed in this specification is a critical medical device and security is of the paramount importance. To control access to the device and the system has two critical security features the first is that all connected devices are dedicated devices which are designed to interact only with the device and that the dedicated devices has a three-step security system of a username, password and at least one of the following.

The system can also utilize a security system based on a GPS location of the user device attempting to communicate with the system. The device signing into the system or client node sends the GPS coordinates of the client node to the receiving device and the receiving device compares the GPS coordinates of the client node to those coordinates that reside in a database of sanctioned locations on the receiving device. If there GPS coordinates from the client node match a set of GPS coordinates in the database of sanctioned locations the client node is allowed to log into the system and the receiving device allows access to the system if the GPS coordinates and other log in credentials are correct. The log in credentials can include a username and password as well as a pin. Some systems may provide additional security by issuing a single use pin to the user through the use of SMS text messaging or email.

An alternative security system can consist of a database of sanctioned IP addresses of permitted client nodes on the receiving system. This database is used to confirm that the client's node trying to access the system is actually a sanctioned device. The security system works as follows the client node sends the IP address to the receiving device and the receiving device checks the IP address is in a database of sanctioned IP addresses on the receiving device sanctioned IP address database. If the IP address of the client node is in the database of sanctioned IP addresses on the receiving device then then the client node is allowed to log into the receiving device if the IP address is correct and other log in credentials are correct. The log in credentials can include a username and password as well as a pin. Some systems may provide additional security by issuing a single use pin to the user through the use of SMS text messaging or email.

The use of the dedicated device places a second processor between the surgical instrument and any interaction with the cloud thereby ensuring security of the surgical instrument and the safety of the patient.

A number of implementations have been described. Nevertheless, it will be understood that various modifications may be made without departing from the spirit and scope of the disclosure. Accordingly, other implementations are within the scope of the following claims.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

I claim:

1. A surgical instrument, comprising:
a handle assembly including a housing, a trigger movably coupled to the housing, a joystick movably coupled to the housing, a mode selection interface disposed on an exterior surface of the housing, and a microcontroller unit (MCU) disposed within the housing;
an elongate shaft extending distally from the handle assembly; and
an end effector assembly disposed at a distal end of the elongate shaft, wherein the MCU is operably coupled to the trigger, the joystick, and the mode selection interface, and is configured to receive input signals from the trigger, the joystick, and the mode selection interface, and to control operation of the surgical instrument based on the received input signals and a selected operative mode.

2. The surgical instrument of claim 1, wherein the trigger is configured to detect user-applied displacement in inward, outward, and lateral directions.

3. The surgical instrument of claim 1, wherein the joystick is configured to detect user-applied displacement in pitch, yaw, and depression directions.

4. The surgical instrument of claim 1, wherein the mode selection interface comprises a plurality of buttons, each button corresponding to a distinct operative mode.

5. The surgical instrument of claim 4, wherein the plurality of operative modes includes a normal mode, a suction mode, an irrigation mode, and an electrosurgical mode.

6. The surgical instrument of claim 5, wherein the MCU is configured to dynamically reassign functions of the trigger and the joystick based on the selected operative mode.

7. The surgical instrument of claim 6, further comprising at least one Hall effect sensor operably coupled to the trigger and configured to detect displacement of the trigger, and at least one Hall effect sensor operably coupled to the joystick and configured to detect displacement of the joystick.

8. A method of operating a surgical instrument, comprising:
receiving, by a microcontroller unit (MCU), a mode selection input from a mode selection interface disposed on an exterior surface of a handle assembly;
receiving, by the MCU, a trigger input signal from a trigger movably coupled to the handle assembly;
receiving, by the MCU, a joystick input signal from a joystick movably coupled to the handle assembly; and
controlling, by the MCU, operation of an end effector assembly disposed at a distal end of an elongate shaft extending from the handle assembly based on the received mode selection input, trigger input signal, and joystick input signal.

9. The method of claim 8, wherein the mode selection input corresponds to one of a normal mode, a suction mode, an irrigation mode, and an electrosurgical mode.

10. The method of claim 9, further comprising dynamically reassigning, by the MCU, functions of the trigger and the joystick based on the received mode selection input.

11. The method of claim 10, wherein the trigger input signal corresponds to user-applied displacement of the trigger in inward, outward, and lateral directions.

12. The method of claim 11, wherein the joystick input signal corresponds to user-applied displacement of the joystick in pitch, yaw, and depression directions.

13. The method of claim 12, further comprising:
detecting, by at least one Hall effect sensor, displacement of the trigger; and
detecting, by at least one Hall effect sensor, displacement of the joystick.

14. The method of claim 13, wherein controlling operation of the end effector assembly comprises:
activating a fluid flow through the elongate shaft when in the suction mode or the irrigation mode;
articulating the end effector assembly based on the joystick input signal; and
actuating the end effector assembly based on the trigger input signal.

15. A surgical system, comprising:
a surgical instrument including a handle assembly, an elongate shaft extending distally from the handle assembly, and an end effector assembly disposed at a distal end of the elongate shaft;
a fluid management subsystem operably coupled to the surgical instrument; and
an energy delivery subsystem operably coupled to the surgical instrument,
wherein the handle assembly includes a housing, a trigger movably coupled to the housing, a joystick movably coupled to the housing, a mode selection interface disposed on an exterior surface of the housing, and a microcontroller unit (MCU) disposed within the housing, the MCU configured to control the fluid management subsystem and the energy delivery subsystem based on inputs received from the trigger, the joystick, and the mode selection interface.

16. The surgical system of claim 15, wherein the fluid management subsystem comprises at least one solenoid valve operably coupled to the MCU and configured to control fluid flow through the elongate shaft based on the selected operative mode and input signals received from the trigger.

17. The surgical system of claim 16, wherein the energy delivery subsystem comprises an electrosurgical generator operably coupled to the MCU and configured to deliver electrosurgical energy to the end effector assembly based on input signals received from the joystick.

18. The surgical system of claim 17, wherein the end effector assembly comprises a pair of jaw members movable between an open position and a closed position, and wherein the MCU is configured to control movement of the jaw members based on input signals received from the trigger.

19. The surgical system of claim 18, wherein the elongate shaft includes at least one fluid channel extending therethrough, and wherein the MCU is configured to control fluid flow through the at least one fluid channel based on the selected operative mode.

20. The surgical system of claim 19, further comprising a gearbox assembly removably coupled to the handle assembly, the gearbox assembly including a plurality of pulleys operably coupled to a plurality of motors disposed within the housing, wherein the MCU is configured to control the plurality of motors to actuate the end effector assembly based on input signals received from the trigger and the joystick.
